(19) United States
(12) Patent Application Publication
(10) Pub. No.: US 2014/0220000 A1
(43) Pub. Date: Aug. 7, 2014

(54) MHC ENGAGEMENT AND CLIP MODULATION FOR THE TREATMENT OF DISEASE

(71) Applicants: Martha Karen Newell, Holland, TX (US); Cassie L. Harvey, Salado, TX (US); Richard Tobin, Aurora, CO (US)

(72) Inventors: Martha Karen Newell, Holland, TX (US); Cassie L. Harvey, Salado, TX (US); Richard Tobin, Aurora, CO (US)

(73) Assignees: Scott & White Healthcare, Temple, TX (US); The Texas A&M University System, College Station, TX (US)

(21) Appl. No.: 14/009,944

(22) PCT Filed: Apr. 4, 2012
(86) PCT No.: PCT/US12/32095

§ 371 Date: Mar. 18, 2014

Related U.S. Application Data
(60) Provisional application No. 61/471,426, filed on Apr. 4, 2011.

(51) Int. Cl. A61K 39/395; A61K 31/713; A61K 38/10

(52) U.S. Cl. CPC A61K 39/3955; A61K 38/10; A61K 31/713; A61K 2039/505
USPC 424/133.1; 424/172.1; 435/375

(57) ABSTRACT

The invention relates to methods for treating disorders by targeting CLIP molecules and MHC. The methods are useful for treating, inhibiting the development of, or otherwise dealing with diseases such as HIV, blood vessel proliferative disorder, cancer and fibrosis.

### RELATED APPLICATION

This application claims the benefit under 35 U.S.C. §119(e) of U.S. provisional application serial number U.S. 61/471,426, entitled “MHC ENGAGEMENT AND CLIP MODULATION FOR THE TREATMENT OF DISEASE” filed on Apr. 4, 2011, the disclosure of which is incorporated by reference herein in its entirety.

### BACKGROUND OF INVENTION

The immune system consists of innate and adaptive or acquired immune responses. The innate immune response is immediate and includes physical, chemical or mechanical barriers, along with cells that are the first to the site of infection or damage, the neutrophils, followed by dendritic cells and macrophages, all of which can engulf or phagocytize potentially harmful pathogens or debris. The phagocytes also are involved in the transition between innate and acquired immunity by processing and loading their engulfed material, loading the fragments into molecules that are embedded in the lysosomal/endosomal compartments, the Major and Minor Histocompatibility Complex (MHC) encoded molecules of T cell immunity. MHC is believed to be the genetic complex responsible for the rejection. The MHC genes, also known as immune response or IR genes, and their protein products are responsible for all graft rejection. There are two types of MHC molecules: MHC class I and MHC class II. All nucleated cells express cell surface MHC class I. A subset of specialized cells express class II MHC. Included in the specialized, professional antigen-presenting cells (APCs) are B cells, macrophages, microglia, dendritic cells, and Langerhans cells among others.

B lymphocytes are specialized cells with specific receptors that are antigen specific that ultimately secrete a soluble copy of its membrane-bound antigen receptor. Once antigen has been bound by the antigen receptor on the B cell, the antigen and its receptor are engulfed into an endosomal compartment. This compartment fuses with another compartment known as the lysosome. The B cell is very efficient at breaking down antigens into smaller parts and loading the parts into MHC class II in the lysosome. The MHC is then trafficked to the cell surface where the B cell can effectively “show” the antigen to a CD4+ T cell. The activated CD4 cell is also called a helper cell and there are two major categories, Th1 and Th2.

The MHC molecules are tightly protected in the endosomal/lysosomal compartments to insure that only antigens for which we need a response get presented to T cells. MHC class II molecules, prior to antigen loading, are associated with a molecule called invariant chain, also known as CD74. The invariant chain is associated with MHC class II (and recently shown to be associated with certain MHC class I molecules) prior to antigen loading into the antigen binding grooves of the MHC molecules. As antigen is processed, the invariant chain gets cleaved by proteases within the compartment. First an end piece is removed, and then another known as CLIP (class II invariant chain associated peptide). CLIP fills the groove that will ultimately hold the antigen until the antigen is properly processed. For a detailed review of the invariant chain, including CLIP, see Matza et al. (2003), incorporated herein in its entirety. Despite the fact that this “chaperone” role for invariant chain and CLIP has been identified, the full impact of these molecules on immune signaling and activation has yet to be determined.

### SUMMARY OF INVENTION

The invention is based at least in part on the discovery that CLIP on the cell surface provides a protective “armor” for a cell expressing cell surface CLIP and that CLIP in the groove of MHC class II of cells, such as cells in the gastrointestinal tract, not only prevents T cell recognition, but also provides protection from MHC Class II or MHC class I mediated cell death. It has been found that CLIP in the groove of MHC class II can directly prevent MHC-mediated cell death. Therefore agents that promote CLIP on MHC expressing cells are useful in the treatment of disorders such as autoimmune disease. For example, the autoimmune disease may be an autoimmune disease involving the GI tract. Conversely, the removal of CLIP from MHC in combination with engagement of MHC with a variety of MHC binding molecules is used to induce death of unwanted cells, for instance, for the treatment of cancer and infectious diseases such as viral disease (e.g., HIV).

In one aspect the invention is a method of treating a subject having hyperproliferative disease by administering to the subject a MHC class II specific CLIP inhibitor and an MHC binding agent in an effective amount to treat the subject. In another aspect the invention is a method of treating a subject having cancer by administering to the subject a MHC class II specific CLIP inhibitor and an MHC binding agent in an effective amount to treat the subject. In another aspect the invention is a method of treating a subject by identifying a subject having an MHC positive cancer and administering to the subject a CLIP inhibitor and an MHC binding agent in an effective amount to treat the subject.

In some embodiments the cancer is a gastrointestinal cancer or a cervical cancer. In other embodiments the cancer is a cancer associated with myeloid suppressor cells, such as a glioblastoma, a colon carcinoma, or a pancreatic cancer.

The MHC binding agent in some embodiments is an anti-MHC class II antibody and in other embodiments is an anti-MHC class I antibody.

The CLIP inhibitor may be synthetic. In some embodiments the CLIP inhibitor is a thymus nuclear protein extract (TNP peptide).

A method of inducing MHC class I or MHC class II-mediated cell death is provide in other aspects of the invention. The method involves contacting an MHC expressing cell, wherein the MHC expressing cell is not a B cell, with a CLIP inhibitor in an effective amount to remove endogenous CLIP peptide from the cell surface and contacting the cell with an MHC binding agent to induce MHC class I or MHC class II-mediated cell death. In some embodiments the MHC binding agent is a MHC class I binding agent and in other embodiments it is a MHC class II binding agent.

The CLIP inhibitor in some embodiments is synthetic. In some embodiments the CLIP inhibitor is an siRNA. The siRNA may be a CLIP siRNA. In other embodiments the CLIP inhibitor is a TNP peptide. In some embodiments the MHC expressing cell is in a subject and the CLIP inhibitor and MHC binding agent are administered to a subject. In some embodiments the subject has a viral infection and the CLIP inhibitor and MHC binding agent are administered to the subject systemically. For instance the subject may have HIV. The CLIP inhibitor and MHC binding agent may be administered in an effective amount to kill HIV-infected, MHC expressing CD4+ T cells or CD4+ macrophages. In other embodiments the subject has a cancer such as a gastrointestinal cancer or a cervical cancer.

A method of protecting a cell from MHC class I or MHC class II-mediated cell death is also provided according to aspects of the invention. The method involves contacting an MHC expressing cell, wherein the MHC expressing cell is not a B cell, with a CLIP inducing agent in an effective amount to place CLIP peptide on the cell surface and protect the cell from MHC class I or MHC class II-mediated cell death.

In some embodiments the MHC binding agent is a MHC class I binding agent and in other embodiments it is a MHC class II binding agent.

The MHC expressing cell in some embodiments is an epithelial cell, such as a gastrointestinal epithelial cell, an oral mucosal epithelial cell, a cervical epithelial cell, or a vaginal epithelial cell. In other embodiments the MHC expressing cell is an endothelial cell.

In some embodiments the CLIP inducing agent is exogenous CLIP. For instance, the exogenous CLIP may be an amino acid sequence comprising a region consisting essentially of SEQ ID NO:1 (Met Arg Met Ala Thr Pro Leu Leu Met).

The MHC expressing cell in some embodiments is in a subject and the CLIP inducing agent is administered to a subject. The subject may have a viral infection and in some embodiments the CLIP inducing agent is administered to the subject orally. Optionally, the subject may be systemically administered a CLIP inhibitor. In some embodiments the viral infection is caused by HIV or H. pylori. In other embodiments the subject has an autoimmune disease and the CLIP inducing agent is administered to the subject orally. The autoimmune disease may be, for instance, Crohn's disease or ulcerative colitis.

In another aspect the invention is a method of treating a subject having mesothelioma by administering to the subject an autophagy inhibitor and an anti-VEGF antibody in an effective amount to treat the subject. In some embodiments the anti-VEGF antibody is bevacizumab.

A method of treating a subject having breast cancer is provided according to other aspects of the invention. The method involves administering to the subject an autophagy inhibitor and a taxane in addition to a CLIP inhibitor and/or a MHC binding agent in an effective amount to treat the subject.

In some embodiments the methods further involve administering to the subject a CLIP inhibitor and/or an MHC binding agent.

The autophagy inhibitor in some embodiments is a 4-aminoquinoline or a pharmaceutically acceptable salt or prodrug thereof. The autophagy inhibitor may be, for instance, chloroquine, 2-hydroxychloroquine, amodiaquine, mondesethylchloroquine, quinoline phosphate, or chloroquine phosphate or mixtures thereof.

A composition of an autophagy inhibitor, a CLIP inhibitor and an MHC binding agent is provided according to other aspects of the invention. Optionally, the composition further includes a carrier.

A kit is provided according to other aspects of the invention. The kit includes one or more containers housing an autophagy inhibitor, a CLIP inhibitor and an MHC binding agent and instructions for administering to a subject the autophagy inhibitor, the CLIP inhibitor and the MHC binding agent.

In other aspects the invention is any of the compositions or combinations of compositions described herein for use in the treatment of cancer or in the manufacture of a medicament for the treatment of cancer.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 depicts % cell death in either untreated CRL5822 cells or cells treated with LPS, MKR.4, MKR.4+LPS, anti-MHC class II antibody, LPS+anti-MHC class II antibody, MKR.4+anti-MHC class II antibody or MKR.4+LPS+anti-MHC class II antibody.

FIG. 2 depicts % cell death in either untreated C57BL/6 splenocyte cells or cells treated with CpG, MKR.4, MKR.4+CpG, anti-MHC class II antibody, CpG+anti-MHC class II antibody, MKR.4+anti-MHC class II antibody or MKR.4+CpG+anti-MHC class II antibody.

FIG. 3 depicts % cell death in either untreated H69 or WITT cells or H69 or WITT cells treated with CpG, MKR.4, MKR.4+CpG, anti-MHC class II antibody, CpG+anti-MHC class II antibody, MKR.4+anti-MHC class II antibody or MKR.4+CpG+anti-MHC class II antibody (cell viability depicted as hemacytometer/trypan blue counts).

FIG. 4 depicts % cell death in either untreated L1210DDP cells or L1210DDP cells treated with CpG, MKR.4, MKR.2, MKR.4+CpG, MKR.2+CpG, MKR.4+MKR.2+CpG, anti-MHC class II antibody, CpG+anti-MHC class II antibody, MKR.4+anti-MHC class II antibody, MKR.2+anti-MHC class II antibody, MKR.4+CpG+anti-MHC class II antibody, MKR.2+CpG+anti-MHC class II antibody, or MKR.4+MKR.2+CpG+anti-MHC class II antibody (cell viability depicted as hemacytometer/trypan blue counts).

FIG. 5 is a bar graph depicting the response of MCF7 cells to B-estradiol. The MCF7 cells responded to B-estradiol by inducing CLIP expression, where untreated MCF7 cells and estradiol treated and untreated drug resistant MCF7 cells (ADR) did not induce CLIP expression.

FIG. 6 is a set of bar graphs depicting the percent change in cell death of CpG treated isolated B cells from wild type C57B/6 mice and CLIP−/− mice.

FIG. 7 is a bar graph depicting the treatment of Daudi cells with MKR.4 peptide (CLIP Inhibitor), RT2 (low binder peptide for MHC Class II), or Hydroxychloroquine (autophagy inhibitor followed by Anti-HLA-DR, DQ, DQ antibody. The cells were analyzed for cell death as shown in the graph.

FIG. 8 is a bar graph depicting the treatment of Daudi cells with CpG ODN followed by treatment with MKR.4, RT2, or Hydroxychloroquine and then Anti-HLA-DR, DQ, DQ antibody. The cells were then analyzed for cell death as shown in the Figure.

FIG. 9 is a bar graph depicting the treatment of splenocytes collected from a C57BL/6 mouse with MKR.4 or RT2 and followed with an MHC antibody. The amount of cell death was determined.

FIG. 10 is a bar graph depicting the treatment of splenocytes collected from a C57BL/6 mouse with CpG ODN first, followed by MKR.4 or RT2 and then with an MHC antibody. The amount of cell death was determined.

### DETAILED DESCRIPTION

When a B cell is activated non-specifically, we previously discovered that the B cell expresses an important, small self-peptide called MHC class II invariant peptide, CLIP. In most individuals, a control cell, known as a T regulatory cell (Treg for short), has been shown, to kill the activated B cell. If naïve B cell MHC molecules are engaged prior to B cell antigen receptor engagement, the consequence for the B cell is cell death (Newell, et al. PNAS 90 (3) 1127-1131, 1993). It has been shown that products of bacteria, viruses, and parasites cause cell surface expression of MHC class II invariant peptide (CLIP) in the groove of MHC class II and, most likely, MHC class I, over time through cross presentation. Because CLIP is a highly conserved self peptide, no traditional CD4 T cell can recognize CLIP directly in the groove of MHC class II because any CD4 that could have recognized the molecule would have been deleted in the thymus. It has been found that CLIP in the groove of MHC class II on a cell not only would not be recognized by a conventional CD4 or CD8 T cell, but also that CLIP provides protection from MHC Class II or MHC class I mediated cell death when MHC class I or class II are engaged by a variety of ligands. It has been demonstrated experimentally that CLIP in the groove of MHC class II can directly prevent MHC-mediated cell death. Therefore, the invention in some aspects is a method for preventing cell death by the addition of CLIP to MHC to protect MHC expressing cells from dying. Conversely, the removal of CLIP from MHC in combination with engagement of MHC in order to promote cell death is used to induce death of unwanted cells in the gastrointestinal tract and elsewhere, for instance, for the treatment of hyperproliferative disease, such as cancer.

These findings have important implications in the treatment of autoimmune disease, infectious disease, and hyperproliferative diseases.

Many therapies to block autoimmune disease involve eliminating or inhibiting B cells. No one knows the mechanism by which these B cell depleting therapies make people better. As described in co-pending U.S. Ser. No. 12/011,643 filed Jan. 28, 2008, entitled METHODS OF MODULATING IMMUNE FUNCTION, and naming M. Karen Newell, Evan Newell and Joshua Cabrera as inventors, antigen non-specifically activated human B cells were treated with anti-CLIP antibodies and subjected to flow cytometry. It was found that the antigen-non-specifically activated B cells express cell surface CLIP. Thus, the inventors of U.S. Ser. No. 12/011,643 recognized that B cell surface expression of CLIP ultimately resulted in B cells that can present self antigens (antigens that load into the CLIP binding site) to Th1 cells, resulting in autoimmune disease.

A properly functioning immune system must discriminate between self and non-self. Failure of this process causes destruction of cells and tissues of the body in the form of autoimmune disease. Recognition of antigen in the thymus deletes some potentially self-reactive T cells from the repertoire. The process of antigen-specific T cell death in the thymus is commonly referred to as “negative” selection. The CD4⁺ or CD8⁺ T cells that recognize self MHC class I or MHC class II plus self antigen (like CLIP) will be deleted in the thymus. Those that could recognize CLIP and someone else's MHC class I or class II will not have been deleted. The cells that meet all of the survival criterion, e.g. appropriate recognition of antigen and either MHC class I for the developing CD8⁺ T or MHC class II for the developing CD4⁺ T cell travel to other regions of the body.

It has been found that if an MHC molecule on an epithelial or endothelial cell of a mucosal tract is associated with CLIP, the cell will be protected from MHC mediated cell death. In order to reduce self-cell death in a subject having autoimmune disease, the self cells ordinarily targeted for destruction in autoimmune disease can be treated to express CLIP in the context of MHC, such that they are protected from destruction. This may be accomplished by treating the subject with a CLIP inducer to promote expression of the CLIP on the cell surface.

A CLIP inducing agent as used herein refers to a compound that results in increased CLIP molecule presentation on the cell surface in the context of MHC. CLIP inducing agents include, for instance, CLIP expression vectors and CLIP activators. A CLIP expression vector is a vector that when administered to the cells causes production of a CLIP molecule protein. The CLIP molecule protein may be CD74, for instance. In the case that CD74 is produced it is desired that the CD74 be produced in the cell such that it can be processed intracellularly to produce a CLIP associated with MHC. Alternatively it may be processed in other cells that are capable of secreting it such that CD74 protein is capable of interacting with MHC on the surface. The expression vector may also produce a CLIP peptide either intracellularly or extracellularly. CLIP activators include for instance exogenous CLIP, palmitoylated protein or PAM, and an anti-CD40 or CD40L molecule in combination with IL-4.

Conversely, in some embodiments, if an MHC molecule on a cell, such as an epithelial or endothelial cell of for instance the mucosal tract, is devoid of CLIP, the cell will be susceptible to MHC mediated cell death. In order to promote cell death in a subject having cancer or other hyperproliferative disorder, the subject can be administered a CLIP inhibitor in combination with an anti-MHC binding agent. Thus, the invention encompasses a method for treating cancer in a subject by administering a CLIP inhibitor in combination with an anti-MHC binding agent to the subject.

A hyperproliferative disease refers to a disorder involving unwanted cellular proliferation of mammalian cells. The invention involves treatment of such disorders using the compounds of the invention. Mammalian cells treatable in this manner include vascular smooth muscle cells, fibroblasts, endothelial cells, lymphocytes, various pre-cancer and cancer cells. The unwanted cell proliferation is inhibited in a subject suffering from a disorder that is characterized by unwanted or abnormal cell proliferation. Such diseases include but are not limited to cancers, blood vessel proliferative disorders and fibrotic disorders. The different diseases described herein are not necessarily independent. For example, fibrotic disorders may be related to, or overlap with, blood vessel disorders and, atherosclerosis, a blood vessel disorder, results in the abnormal formation of fibrous tissue.

Blood vessel proliferation disorders refer to angiogenic and vasculogenic disorders generally resulting from abnormal proliferation of blood vessels. Examples of such disorders include restenosis, retinopathies, and arteriosclerotic conditions. An arteriosclerotic condition is a disorder involving a thickening and hardening of the arterial wall, for instance, classical atherosclerosis, accelerated atherosclerosis, atherosclerotic lesions, post-angioplastic restenesis, intimal smooth muscle cell proliferation, restenosis, vascular occlusion and any other arteriosclerotic conditions characterized by undesirable endothelial and/or vascular smooth muscle cell proliferation, including vascular complications of diabetes, diabetic glomerulosclerosis and diabetic retinopathy. Examples of other blood vessel proliferative disorders include arthritis and ocular diseases such as diabetic retinopathy and age related macular degeneration (AMD).

Fibrotic disorders may be due to the abnormal formation of an extracellular matrix. Examples of fibrotic disorders include hepatic cirrhosis, mesangial proliferative cell disorders and skin disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Mesangial disorders are brought about by abnormal proliferation of mesangial cells. Mesangial hyper-proliferative cell disorders include various human renal diseases, such as glomerulonephritis, diabetic nephropathy, malignant nephrosclerosis, thrombotic microangiopathy syndromes, transplant rejection, and glomerulopathies.

Benign hyperproliferative disorders of the skin result typically from excess keratin deposition (hyperkeratosis) of the corneous layer. Such hyperproliferative disorders include but are not limited to epidermolytic hyperkeratosis, keloid, dermatitis papillaris capilliti (acne keloid), fibromatosis gingivae (keloid of gums), epidermolytic hyperkeratosis, follicular keratosis, and hypertrophic scar formation. Malignant hyperproliferative disorders of the skin include but are not limited to Kaposi's sarcoma and skin cancer (e.g. basal cell carcinoma, squamous cell carcinoma, melanoma).

In some aspects the present invention provides a method of treating a hyperproliferative disease that is a cancer of the gastrointestinal system by rendering the cancer susceptible to cell death. The method involves administering to a subject by an oral route a therapeutically effective amount of a composition comprising a CLIP inhibitor. A composition of the invention may, for example, be used as a first, second, third or fourth line cancer treatment. In some embodiments, the invention provides methods for treating a cancer (including ameliorating of a symptom thereof) in a subject refractory to one or more conventional therapies for such a cancer, said methods comprising administering to said subject a therapeutically effective amount of a composition comprising a CLIP inhibitor.

While not bound by a specific mechanism of action, it is believed that the CLIP inhibitors of the invention displace CLIP from MHC class I leaving the cell susceptible to death. The invention also involves administering another anti-cancer treatment (e.g., radiation therapy, chemotherapy or surgery) to a subject. Examples of conventional cancer therapies include treatment of the cancer with agents such as All-trans retinoic acid, Actinomycin D, adriamycin, anastrozole, Azacitidine, Azathioprine, Alkeran, Ara-C, Arsenic Trioxide (Trisenox), Avastin, BiCNU Bleomycin, Busulfan, CCNU, Carboplatin, Capecitabine, Cisplatin, Chlorambucil, Cyclophosphamide, Cytarabine, Cytoxan, DTIC, Daunorubicin, Docetaxel, Doxifluridine, Doxorubicin, 5-fluorouracil, Epirubicin, Epothilone, Etoposide, exemestane, Erlotinib, Fludarabine, Fluorouracil, Gemcitabine, Hydroxyurea, Herceptin, Hydrea, Ifosfamide, Irinotecan, Idarubicin, Imatinib, letrozole, Lapatinib, Leustatin, 6-MP, Mithramycin, Mitomycin, Mitoxantrone, Mechlorethamine, megestrol, Mercaptopurine, Methotrexate, Mitoxantrone, Navelbine, Nitrogen Mustard, Oxaliplatin, Paclitaxel, pamidronate disodium, Pemetrexed, Rituxan, 6-TG, Taxol, Topotecan, tamoxifen, taxotere, Teniposide, Tioguanine, toremifene, trimetrexate, trastuzumab, Valrubicin, Vinblastine, Vincristine, Vindesine, Vinorelbine, Velban, VP-16, and Xeloda.

Cancer therapies and their dosages, routes of administration and recommended usage are known in the art and have been described in such literature as the Physician's Desk Reference (56^{th} ed., 2002). In some embodiments, the CLIP inhibitor is formulated into a pharmaceutical composition that further comprises one or more such anticancer agents.

Cancers that can be treated by the methods encompassed by the invention include, but are not limited to, neoplasms, malignant tumors, metastases, or any disease or disorder characterized by uncontrolled cell growth such that it would be considered cancerous. The cancer may be a primary or metastatic cancer. Specific cancers that can be treated according to the present invention include, but are not limited to, those listed below (for a review of such disorders, see Fishman et al., 1985, Medicine, 2d Ed., J. B. Lippincott Co., Philadelphia).

Cancers include, but are not limited to, gastrointestinal cancers, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor.

Gastrointestinal cancers include, but are not limited to, benign esophageal tumors, colorectal cancer, esophageal cancer, gastrointestinal stromal tumors, pancreatic cancer, pancreatic endocrine tumors, polyps of the colon and rectum, small bowel tumors and stomach cancer.

Colorectal cancer is a common cancer that is treated by surgical resection and chemotherapy (typically 5-fluorouracil and leucovorin, but also capecitabine (a 5-fluorouracil precursor), irinotecan, and oxaliplatin) as well as with monoclonal antibodies such as bevacizumab, cetuximab, and panitumumab. Another type of cancer, anorectal cancer typically is treated by surgery and combination chemotherapy and radiation therapy. Esophageal cancer includes, for example, adenocarcinoma spindle cell carcinoma, verrucous carcinoma, pseudosarcoma, mucoepidermoid carcinoma, adenosquamous carcinoma, cylindroma, primary oat cell carcinoma, choriocarcinoma, carcinoid tumor, sarcoma, and primary malignant melanoma. Treatment generally includes surgery combined with radiation and chemotherapy such as, cisplatin, 5-fluorouracil, mitomycin, doxorubicin, vindesine, bleomycin, and methotrexate. Gastrointestinal stromal tumors occur in the stomach, the small bowel, the esophagus, the colon, and the rectum. Treatment involves surgery and possible administration of the tyrosine kinase inhibitor imatinib.

Pancreatic cancer includes for instance, gastrointestinal stromal tumors (GIST, tumors of the GI tract derived from mesenchymal precursor cells in the gut wall). Traditional therapy involves surgery and 5-fluorouracil (5-FU), gemcitabine, irinotecan, paclitaxel, oxaliplatin, carboplatin with or without external beam radiation.

Pancreatic endocrine tumors include for instance, insulinoma, vipoma and glucagonoma. Streptozotocin may be used, either alone or in combination with 5-fluorouracil (5-FU) or doxorubicin or chlorozotocin and interferon for the treatment of Pancreatic endocrine tumors. Insulinoma is a rare pancreatic β-cell tumor that hypersecretes insulin. Drugs that block insulin secretion (e.g., diazoxide, octreotide, Ca channel blockers, β-blockers, phenyloin) may be used for treatment in addition to surgery. Vipoma is a non-β pancreatic islet cell tumor secreting vasoactive intestinal peptide (VIP). Glucagonoma is a pancreatic α-cell tumor that secretes glucagon, causing hyperglycemia and a characteristic skin rash.

Small-Bowel Tumors include benign tumors such as leiomyomas, lipomas, neurofibromas, and fibromas. Adenocarcinoma, primary malignant lymphoma arising in the ileum, carcinoid tumors, and Kaposi's sarcoma.

Stomach cancer is caused in some instance by Helicobacter pylori. Therapy typically involves surgery, sometimes combined with chemotherapy (5-fluorouracil, doxorubicin, mitomycin, cisplatin, or leucovorin in various combinations), radiation, or both.

A cancer may be determined to be refractory to a therapy when at least some significant portion of the cancer cells are not killed or their cell division are not arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased.

A CLIP inhibitor as used herein is any molecule that reduces the association of a CLIP molecule with MHC by binding to the MHC and blocking the CLIP-MHC interaction. The CLIP inhibitor may function by displacing CLIP from the surface of a CLIP molecule expressing cell. A CLIP molecule expressing cell is a cell that has MHC class I or II on the surface and includes a CLIP molecule within that MHC. Such cells include, for example, intestinal epithelial cells, endothelial cells, epithelial cells lining the uterine wall, and skin cells.

The CLIP molecule, as used herein, refers to intact CD74 (also referred to as invariant chain) or intact CLIP, as well as the naturally occurring proteolytic fragments thereof. Intact CD74 or intact CLIP refer to peptides having the sequence of the native CD74 or native CLIP respectively. The CLIP molecule is one of the naturally occurring proteolytic fragments of CD74 or CLIP in some embodiments. The CLIP molecule may be, for example, at least 90% homologous to the native CD74 or CLIP molecules. In other embodiments the CLIP molecule may be at least 91%, 92%, 93%, 94%, 95%, 96%, 97%, 98%, or 99% homologous to the native CD74 or CLIP molecules An example of native CLIP molecule is MRMATPLLM (SEQ ID NO: 1), and in three-letter abbreviation as: Met Arg Met Ala Thr Pro Leu Leu Met (SEQ ID NO: 1). An example of native CD74 molecule is MHRRRSRSCR EDQKPVMDDQ RDLISNNEQL PMLGRRPGAP ESKCSRGALY TGFSILVTLL AGQATTAYF LYQQQGRLDK LTVTSQNLQL ENLRMKLPKP PKPVSKMRMA TPLLMQALPM GALPQGPMQN ATKYGNMTED HVMHLLQNAD PLKVYPPLKG SFPENLRHLK NTMETIDWKV FESWMHHWLL FEMSRHSLEQ KPTDAPPKVL TKCQEEVSHI PAVHPGSFRP KCDENGNYLP LQCYGSIGYC WCVFPNGTEV PNTRSRGHHN CSESLELEDP SSGLGVTKQD LGPVPM (SEQ ID NO 2).

The function of the CLIP molecule in this invention is mainly as an MHC class I or MHC class II chaperone and protective shield. MHC class II molecules are heterodimeric complexes that present foreign antigenic peptides on the cell surface of antigen-presenting cells (APCs) to CD4⁺ T cells. MHC class II synthesis and assembly begins in the endoplasmic reticulum (ER) with the non-covalent association of the MHC α and β chains with trimers of CD74. CD74 is a non-polymorphic type II integral membrane protein; murine CD74 has a short (30 amino acid) N-terminal cytoplasmic tail, followed by a single 24 amino acid transmembrane region and an ˜150 amino acid long lumenal domain. Three MHC class II αβ dimers bind sequentially to a trimer of the CD74 to form a nonameric complex (αβIi)3, which then exits the ER. After being transported to the trans-Golgi, the αβIi complex is diverted from the secretory pathway to the endocytic system and ultimately to acidic endosome or lysosome-like structures called MHC class I or II compartments.

The N-terminal cytoplasmic tail of CD74 contains two extensively characterized dileucine-based endosomal targeting motifs. These motifs mediate internalization from the plasma membrane and from the trans-Golgi network. In the endocytic compartments, the CD74 chain is gradually proteolytically processed, leaving only a small fragment, the class II-associated CD74 chain peptide (CLIP), bound to the released αβ dimers. The final step for MHC class II expression requires interaction of αβ-CLIP complexes with another class II-related αβ dimer, called HLA-DM in the human system. This drives out the residual CLIP, rendering the αβ dimers ultimately competent to bind antigenic peptides, which are mainly derived from internalized antigens and are also delivered to the endocytic pathway. The peptide-loaded class II molecules then leave this compartment by an unknown route to be expressed on the cell surface and surveyed by CD4⁺ T cells.

CLIP inhibitors include peptides and small molecules that can replace CLIP. In some embodiments the CLIP inhibitor is a peptide. A number of peptides useful for displacing CLIP molecules are described in U.S. patent application Ser. No. 12/508,543 (publication number US-2010-0166782-A1); Ser. No. 12/739,459 and Ser. No. 12/508,532 (publication number US-2010-0166789-A1) each of which is herein specifically incorporated by reference. For instance a number of these peptides are “thymus nuclear protein (TNP)” peptides.

CLIP inhibitors include for instance but are not limited to competitive CLIP fragments, MHC class II binding peptides and peptide mimetics. Thus, the CLIP inhibitor includes peptides and peptide mimetics that bind to MHC class II and displace CLIP. For instance, an isolated peptide comprising X₁RX₂X₃X₄X₅LX₆X₇ (SEQ ID NO: 3), wherein each X is an amino acid, wherein R is Arginine, L is Leucine and wherein at least one of X₂ and X₃ is Methionine, wherein the peptide is not N-MRMATPLLM-C (SEQ ID NO: 4), and wherein the peptide is a CLIP displacer is provided according to the invention. X refers to any amino acid, naturally occurring or modified. In some embodiments the Xs referred to the in formula X₁RX₂X₃X₄X₅LX₆X₇ (SEQ ID NO: 7) have the following values:

X₁ is Ala, Phe, Met, Leu, Be, Val, Pro, or Trp

X₂ is Ala, Phe, Met, Leu, Be, Val, Pro, or Trp

X₃ is Ala, Phe, Met, Leu, Be, Val, Pro, or Trp.

wherein X₄ is any

X₅ is Ala, Phe, Met, Leu, Be, Val, Pro, or Trp

X₆ is any

X₇ is Ala, Cys, Thr, Ser, Gly, Asn, Gln, or Tyr.

The peptide preferably is FRIM X₄VLX₆S (SEQ ID NO: 6), such that X₄ and X₆ are any amino acid and may be Ala. Such a peptide is referred to as FRIMAVLAS (SEQ ID NO: 5).

The minimal peptide length for binding HLA-DR is 9 amino acids. However, there can be overhanging amino acids on either side of the open binding groove. For some well studied peptides, it is known that additional overhanging amino acids on both the N and C termini can augment binding. Thus the peptide may be 9 amino acids in length or it may be longer. For instance, the peptide may have additional amino acids at the N and/or C terminus. The amino acids at either terminus may be anywhere between 1 and 100 amino acids. In some embodiments the peptide includes 1-50, 1-20, 1-15, 1-10, 1-5 or any integer range there between. When the peptide is referred to as, e.g., “N—FRIMAVLAS-C” or “N—X₁RX₂X₃X₄X₅LX₆X₇—C” the —C and —N refer to the terminus of the peptide and thus the peptide is only 9 amino acids in length. However the 9 amino acid peptide may be linked to other non-peptide moieties at either the —C or —N terminus or internally.

Other peptides useful as CLIP inhibitors, including some TNP peptides and synthetic peptides are shown in Table 1.

TABLE 1

| Amino Acid | | | |
|---|---|---|---|
| Sequence | SEQ ID NO. | Amino Acid Sequence | SEQ ID NO. |
| LVQNDTLLQ | 10 | FVNDIFERI | 43 |
| VVSTQTALA | 11 | LPNIQAVLL | 44 |
| IMNSFVNDI | 12 | LLPGELAKH | 45 |
| MGIMKSFVN | 13 | FVAFVDKCC | 46 |
| MGIMNSFVN | 14 | LKPDPNTLC | 47 |
| VLIAFSQYL | 15 | MENFVAFVD | 48 |
| IMNSFVNDL | 16 | LFGDELCKV | 49 |
| IMKSFVNDI | 17 | VTIAQGGVL | 50 |
| IQGITKPAI | 18 | MKSFVNDIF | 51 |
| VTAMDVVYA | 19 | LFTFHADIC | 52 |
| YGFQNALIV | 20 | FVNDLFERL | 53 |
| LVNELTEFA | 21 | IAQGGVLPN | 54 |
| FQNALIVRY | 22 | LGSFLYEYS | 55 |
| MSIMNSFVN | 23 | FVDKCCAAD | 56 |
| LVLIAFSQY | 24 | LFEDTNLCA | 57 |
| VQNDTLLQV | 25 | VNFAEFSKK | 58 |
| MGNMNSFVN | 26 | MNSFVNDIF | 59 |
| FQSAIKLVD | 27 | MNSFVNDLF | 60 |
| VAFVDKCCA | 28 | LVDEPQNLI | 61 |
| LVVSTQTAL | 29 | MDVVYALKR | 62 |
| VFLENVIRD | 30 | LLLPGELAK | 63 |
| LIAFSQYLQ | 31 | LTPDETYVP | 64 |
| FQSAAIGAL | 32 | LQNEIDVSS | 65 |
| MDIMNSFVN | 33 | LVDFQDAKA | 66 |
| IKLVDFQDA | 34 | VGLFEDTNL | 67 |
| VMENFVAFV | 35 | LGLIYEETR | 68 |
| YLQQCPFDE | 36 | ILGLIYEET | 69 |
| VLPNIQAVL | 37 | IDVSSREKS | 70 |
| VEPSDTIEN | 38 | LHTLFGDEL | 71 |
| FFQSAIKLV | 39 | LVGLFEDTN | 72 |
| IQAVLLPKK | 40 | IAQDFKTDL | 73 |
| IAFSQYLQQ | 41 | FHADICTLP | 74 |
| FLGSFLYEY | 42 | | |

In some instances the peptides may be mixed with cystatin A and/or histones and in other instances the composition is free of cystatin A or histones. Histone encompasses all histone proteins including H1, H2A, H2B, H3, H4 and H5.

The peptide may be cyclic or non-cyclic. Cyclic peptides in some instances have improved stability properties. Those of skill in the art know how to produce cyclic peptides.

The peptides may also be linked to other molecules. The two or more molecules may be linked directly to one another (e.g., via a peptide bond); linked via a linker molecule, which may or may not be a peptide; or linked indirectly to one another by linkage to a common carrier molecule, for instance.

Thus, linker molecules (“linkers”) may optionally be used to link the peptide to another molecule. Linkers may be peptides, which consist of one to multiple amino acids, or non-peptide molecules. Examples of peptide linker molecules useful in the invention include glycine-rich peptide linkers (see, e.g., U.S. Pat. No. 5,908,626), wherein more than half of the amino acid residues are glycine. Preferably, such glycine-rich peptide linkers consist of about 20 or fewer amino acids.

The peptide for instance, may be linked to a PEG molecule. Such a molecule is referred to as a PEGylated peptide.

In certain embodiments, the CLIP inhibitor is an inhibitory nucleic acid such as a small interfering nucleic acid molecule such as antisense, RNAi, or siRNA oligonucleotide to reduce the level of mature CLIP molecule (CD74) expression. The nucleotide sequences of CD74 molecules are well known in the art and can be used by one of skill in the art using art recognized techniques in combination with the guidance set forth herein to produce the appropriate siRNA molecules. An example of a CD74 nucleic acid molecule is shown in SEQ ID NO. 75:

ccggggggtc agggtcccag atgcacagga ggagaagcag
gagctgtcgg gaagatcaga agccagtcat ggatgaccag
cgcgacctta tctccaacaa tgagcaactg cccatgctgg
gccggcgccc tggggccccg gagagcaagt gcagccgcgg
agccctgtac acaggctttt ccatcctggt gactctgctc
ctcgctggcc aggccaccac cgcctacttc ctgtaccagc
agcagggccg gctggacaaa ctgacagtca cctcccagaa
cctgcagctg gagaacctgc gcatgaagct tcccaagcct
cccaagcctg tgagcaagat gcgcatggcc accccgctgc
tgatgcaggc gctgcccatg ggagccctgc cccaggggcc
catgcagaat gccaccaagt atggcaacat gacagaggac
catgtgatgc acctgctcca gagtcactgg aactggagga
cccgtcttct gggctgggtg tgaccaagca ggatctgggc
ccagtcccca tgtgagagca gcagaggcgg tcttcaacat
cctgccagcc ccacacagct acagctttct tgctcccttc
agcccccagc ccctccccca tctcccaccc tgtacctcat
cccatgagac cctggtgcct ggctctttcg tcacccttgg
acaagacaaa ccaagtcgga acagcagata acaatgcagc
aaggccctgc tgcccaatct ccatctgtca acaggggcgt
gaggtcccag gaagtggcca aaagctagac agatccccgt
tcctgacatc acagcagcct ccaacacaag gctccaagac
ctaggctcat ggacgagatg ggaaggcaca gggagaaggg
ataaccctac acccagaccc caggctggac atgctgactg
tcctctcccc tccagccttt ggccttggct tttctagcct
atttacctgc aggctgagcc actctcttcc ctttccccag
catcactccc caaggaagag ccaatgtttt ccacccataa
tcctttctgc cgacccctag ttccctctgc tcagccaagc
ttgttatcag ctttcagggc catggttcac attagaataa
aaggtagtaa ttagaacaaa aaaaaaaaaa aaaaa

Small interfering nucleic acid (siNA) include, for example: microRNA (miRNA), small interfering RNA (siRNA), double-stranded RNA (dsRNA), and short hairpin RNA (shRNA) molecules. An siNA useful in the invention can be unmodified or chemically-modified. An siNA of the instant invention can be chemically synthesized, expressed from a vector or enzymatically synthesized. Such methods are well known in the art.

In one embodiment, one of the strands of the double-stranded siNA molecule comprises a nucleotide sequence that is complementary to a nucleotide sequence of a target RNA or a portion thereof, and the second strand of the double-stranded siNA molecule comprises a nucleotide sequence identical to the nucleotide sequence or a portion thereof of the targeted RNA. In another embodiment, one of the strands of the double-stranded siNA molecule comprises a nucleotide sequence that is substantially complementary to a nucleotide sequence of a target RNA or a portion thereof, and the second strand of the double-stranded siNA molecule comprises a nucleotide sequence substantially similar to the nucleotide sequence or a portion thereof of the target RNA. In another embodiment, each strand of the siNA molecule comprises about 19 to about 23 nucleotides, and each strand comprises at least about 19 nucleotides that are complementary to the nucleotides of the other strand.

In some embodiments an siNA is an shRNA, shRNA-mir, or microRNA molecule encoded by and expressed from a genomically integrated transgene or a plasmid-based expression vector. Thus, in some embodiments a molecule capable of inhibiting mRNA expression, or microRNA activity, is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

Other inhibitor molecules that can be used include ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat. Med. 4(8):967-71, 1998). For example, neoplastic reversion was obtained using a ribozyme targeted to an H-Ras mutation in bladder carcinoma cells (Feng et al., Cancer Res. 55(10):2024-8, 1995). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9, 1996). Multitarget ribozymes (connected or shotgun) have been suggested as a means of improving efficiency of ribozymes for gene suppression (Ohkawa et al., Nucleic Acids Symp Ser. (29):121-2, 1993).

The methods involve the co-administration of an MHC binding agent such as an anti-MHC antibody with the CLIP inhibitor. The purpose of exposing a cell to an anti-MHC class II antibody, for instance, is to assist in cell death, once CLIP has been removed. Once CLIP has been removed, the antibody will be able to interact with the MHC and cause the cell death. The compounds are co-administered, which means that the compounds may be administered in a single formulation or in separated formulations at the same or different times. Thus, the compounds may be administered simultaneously or sequentially. When the compounds are administered simultaneously they can be administered in the same or separate formulations, but are administered at the same time. The compounds are administered sequentially with one another when the administration of the compounds is temporally separated. The separation in time between the administration of these compounds may be a matter of minutes or it may be longer. In some instances, the CLIP inhibitor is administered prior to the MHC binding agent. As used herein, “prior” means at least, at least three days, at least two days, at least one day, at least six hours, at least five hours, at least four hours, at least three hours, at least two hours, at least one hour, at least 30 minutes, or at least 15 minutes prior to the MHC binding agent.

In some embodiments, the present invention provides a method of treating a hyperproliferative disease comprising administering to a subject in whom such treatment is desired a therapeutically effective amount of a composition comprising a CLIP inhibitor in combination with an anti-MHC binding agent. Non-limiting examples of hyperproliferative disease include cancers, blood vessel proliferative disorders and fibrotic disorders. A composition of the invention may, for example, be used as a first, second, third or fourth line cancer treatment. In some embodiments, the invention provides methods for treating a cancer (including ameliorating a symptom thereof) in a subject refractory to one or more conventional therapies for such a cancer, said methods comprising administering to said subject a therapeutically effective amount of a composition comprising a CLIP inhibitor in combination with an anti-MHC binding agent. A cancer may be determined to be refractory to a therapy when at least some significant portion of the cancer cells are not killed or their cell division are not arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased. Also disclosed are methods of treating a chemoresistant cancer comprising administering to a subject with a chemoresistant cancer a composition comprising a CLIP inhibitor in combination with an anti-MHC binding agent. Also disclosed are methods of inducing chemosensitivity of a cancer comprising administering to a subject with a cancer a CLIP inhibitor in combination with an anti-MHC binding agent. An MHC cancer expressing cell can be detected using methods known in the art, such as antibody detection methods.

MHC binding molecules, also referred to herein as an anti-MHC binding agents, include peptides, antibodies, antibody fragments and small molecules that interact with MHC and cause a MHC signal to be activated. A MHC signal is an intracellular signal that indicates to the cell that MHC has been engaged and causes the cell to undergo cell death. The binding molecules are referred to herein as isolated molecules that selectively bind to MHC. A molecule that selectively binds to MHC as used herein refers to a molecule, e.g., small molecule, peptide, antibody, fragment, that interacts with MHC (also referred to as HLA). MHC-binding regions, in some embodiments derive from the MHC-binding regions of known or commercially available antibodies, or alternatively, they are functionally equivalent variants of such regions.

The term “antibody” herein is used in the broadest sense and specifically covers intact monoclonal antibodies, polyclonal antibodies, multispecific antibodies (e.g. bispecific antibodies) formed from at least two intact antibodies, antibody fragments, so long as they exhibit the desired biological activity, and antibody like molecules such as scFv. A native antibody usually refers to heterotetrameric glycoproteins composed of two identical light (L) chains and two identical heavy (H) chains. Each heavy and light chain has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The peptides useful herein are isolated peptides. As used herein, the term “isolated” means that the referenced material is removed from its native environment, e.g., a cell. Thus, an isolated biological material can be free of some or all cellular components, i.e., components of the cells in which the native material is occurs naturally (e.g., cytoplasmic or membrane component). The isolated peptides may be substantially pure and essentially free of other substances with which they may be found in nature or in vivo systems to an extent practical and appropriate for their intended use. In particular, the peptides are sufficiently pure and are sufficiently free from other biological constituents of their hosts cells so as to be useful in, for example, producing pharmaceutical preparations or sequencing. Because an isolated peptide of the invention may be admixed with a pharmaceutically acceptable carrier in a pharmaceutical preparation, the peptide may comprise only a small percentage by weight of the preparation. The peptide is nonetheless substantially pure in that it has been substantially separated from the substances with which it may be associated in living systems. In some embodiments, the peptide is a synthetic peptide.

The term “purified” in reference to a protein or a nucleic acid, refers to the separation of the desired substance from contaminants to a degree sufficient to allow the practitioner to use the purified substance for the desired purpose. Preferably this means at least one order of magnitude of purification is achieved, more preferably two or three orders of magnitude, most preferably four or five orders of magnitude of purification of the starting material or of the natural material. In specific embodiments, a purified thymus derived peptide is at least 60%, at least 80%, or at least 90% of total protein or nucleic acid, as the case may be, by weight. In a specific embodiment, a purified thymus derived peptide is purified to homogeneity as assayed by, e.g., sodium dodecyl sulfate polyacrylamide gel electrophoresis, or agarose gel electrophoresis.

The MHC binding molecules bind to MHC, preferably in a selective manner. As used herein, the terms “selective binding” and “specific binding” are used interchangeably to refer to the ability of the peptide to bind with greater affinity to MHC and fragments thereof than to non-MHC derived compounds. That is, peptides that bind selectively to MHC will not bind to non-MHC derived compounds to the same extent and with the same affinity as they bind to MHC and fragments thereof, with the exception of cross reactive antigens or molecules made to be mimics of MHC such as peptide mimetics of carbohydrates or variable regions of anti-idiotype antibodies that bind to the MHC-binding peptides in the same manner as MHC. In some embodiments, the MHC binding molecules bind solely to MHC and fragments thereof.

“Isolated antibodies” as used herein refer to antibodies that are substantially physically separated from other cellular material (e.g., separated from cells which produce the antibodies) or from other material that hinders their use either in the diagnostic or therapeutic methods of the invention. Preferably, the isolated antibodies are present in a homogenous population of antibodies (e.g., a population of monoclonal antibodies). Compositions of isolated antibodies can however be combined with other components such as but not limited to pharmaceutically acceptable carriers, adjuvants, and the like.

MHC binding molecules, in some embodiments of the invention, are anti-MHC antibodies, including fragments thereof and small antibody like molecules such as scFv, as well, non-antibody MHC binding peptides, TCR molecules such as soluble T cell receptors (sTCR), single-chain TCRs (scTCR), multimeric TCR (such as tetrameric TCR), and soluble CD4.

TCRs are disulfide linked heterodimers of α and β chain glycoproteins. TCR polypeptides consist of amino terminal variable and carboxy terminal constant regions. While the carboxy terminal region functions as a trans-membrane anchor and participates in intracellular signaling when the receptor is occupied, the variable region is responsible for recognition of antigens.

The TCRs useful according to the invention include sTCR, scTCR, and multimeric TCR such as tetrameric TCR. Molecules such as these are described, for instance, in US Published Patent Application 2008/0125369, which is incorporated by reference. A tetrameric TCR is a multimer of four T cell receptor molecules associated (e.g. covalently or otherwise linked) with one another, preferably via a linker molecule.

In one embodiment, the MHC peptides useful in the invention are isolated intact soluble monoclonal antibodies specific for MHC. As used herein, the term “monoclonal antibody” refers to a homogenous population of immunoglobulins that specifically bind to an identical epitope (i.e., antigenic determinant). In other embodiments, the peptide is an antibody fragment.

The anti-MHC antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biot, 2:593-596 (1992)]. The invention also encompasses the use of single chain variable region fragments (scFv). Single chain variable region fragments are made by linking light and/or heavy chain variable regions by using a short linking peptide.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

The invention also encompasses small molecules that bind to MHC. Such binding molecules may be identified by conventional screening methods, such as phage display procedures (e.g. methods described in Hart et al., J. Biol. Chem. 269:12468 (1994)). Alternatively, binding molecules can be identified from combinatorial libraries. Many types of combinatorial libraries have been described. For instance, U.S. Pat. No. 5,712,171 (which describes methods for constructing arrays of synthetic molecular constructs by forming a plurality of molecular constructs having the scaffold backbone of the chemical molecule and modifying at least one location on the molecule in a logically-ordered array); U.S. Pat. No. 5,962,412 (which describes methods for making polymers having specific physiochemical properties); and U.S. Pat. No. 5,962,736 (which describes specific arrayed compounds).

The invention also includes the treatment of a subject having a cancer after the subject has been diagnosed with an MHC positive cancer. An MHC positive cancer as used herein refers to a cancer that expressed some MHC molecule on the surface. The MHC molecule may be MHC class I or II. The MHC molecule can be detected using any known assay including nucleic acid and protein detection assays. For instance the presence of MHC on a tumor may be assessed using antibodies.

The MHC binding molecules described herein can be used alone for therapeutic or diagnostic aspects of the methods or in conjugates with other molecules such as detection or cytotoxic agents in the detection and treatment methods of the invention, as described in more detail herein.

Typically, one of the components usually comprises, or is coupled or conjugated to a detectable label. A detectable label is a moiety, the presence of which can be ascertained directly or indirectly. Generally, detection of the label involves an emission of energy by the label. The label can be detected directly by its ability to emit and/or absorb photons or other atomic particles of a particular wavelength (e.g., radioactivity, luminescence, optical or electron density, etc.). A label can be detected indirectly by its ability to bind, recruit and, in some cases, cleave another moiety which itself may emit or absorb light of a particular wavelength (e.g., epitope tag such as the FLAG epitope, enzyme tag such as horseradish peroxidase, etc.). An example of indirect detection is the use of a first enzyme label which cleaves a substrate into visible products. The label may be of a chemical, peptide or nucleic acid molecule nature although it is not so limited. Other detectable labels include radioactive isotopes such as P³² or H³, luminescent markers such as fluorochromes, optical or electron density markers, etc., or epitope tags such as the FLAG epitope or the HA epitope, biotin, avidin, and enzyme tags such as horseradish peroxidase, β-galactosidase, etc. The label may be bound to a peptide during or following its synthesis. There are many different labels and methods of labeling known to those of ordinary skill in the art. Examples of the types of labels that can be used in the present invention include enzymes, radioisotopes, fluorescent compounds, colloidal metals, chemiluminescent compounds, and bioluminescent compounds. Those of ordinary skill in the art will know of other suitable labels for the peptides described herein, or will be able to ascertain such, using routine experimentation. Furthermore, the coupling or conjugation of these labels to the peptides of the invention can be performed using standard techniques common to those of ordinary skill in the art.

Another labeling technique which may result in greater sensitivity consists of coupling the molecules described herein to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

Conjugation of the peptides including antibodies or fragments thereof to a detectable label facilitates, among other things, the use of such agents in diagnostic assays. Another category of detectable labels includes diagnostic and imaging labels (generally referred to as in vivo detectable labels) such as for example magnetic resonance imaging (MRI): Gd(DOTA); for nuclear medicine: ²⁰¹Tl, gamma-emitting radionuclide 99mTc; for positron-emission tomography (PET): positron-emitting isotopes, (18)F-fluorodeoxyglucose ((18)FDG), (18)F-fluoride, copper-64, gadodiamide, and radioisotopes of Pb(II) such as 203Pb; 111In.

As used herein, “conjugated” means two entities stably bound to one another by any physiochemical means. It is important that the nature of the attachment is such that it does not impair substantially the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin-avidin and biotin-streptavidin complexation and other affinity interactions. Such means and methods of attachment are well known to those of ordinary skill in the art.

The conjugates also include an antibody conjugated to a cytotoxic agent such as a chemotherapeutic agent, toxin (e.g. an enzymatically active toxin of bacterial, fungal, plant or animal origin, or fragments thereof, or a small molecule toxin), or a radioactive isotope (i.e., a radioconjugate). Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296). Enzymatically active toxins and fragments thereof which can be used in the conjugates include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes.

For selective destruction of the cell, the antibody may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for detection, it may comprise a radioactive atom for scintigraphic studies, for example tc⁹⁹m or I¹²³, or a spin label for nuclear magnetic resonance (NMR) imaging (also known as magnetic resonance imaging, mri), such as iodine-123, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc⁹⁹ⁿ or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The invention also involves the use of CLIP inducing agents to protect an MHC expressing cell from MHC class I or MHC class II mediated cell death. The method is achieved by treating an MHC expressing cell with a CLIP inducing agent to place CLIP on the cell surface in the context of MHC to protect the cell from cell death if the cell is exposed to anti-MHC antibody. Thus, according to the invention, if an MHC molecule on an MHC expressing cell, such as an epithelial or endothelial cell is associated with CLIP, the cell will be protected from cell death. In order to reduce self-cell death in a subject having autoimmune disease, for instance, the self cells ordinarily targeted for destruction in autoimmune disease can be treated to express CLIP in the context of MHC, such that they are protected from destruction. This may be accomplished by treating the subject with a CLIP inducer to promote expression of the CLIP on the cell surface.

A CLIP inducing agent as used herein refers to a compound that results in increased CLIP molecule presentation on the cell surface in the context of MHC. CLIP inducing agents include, for instance, CLIP expression vectors and CLIP activators. A CLIP expression vector is a vector that when administered to the cells causes production of a CLIP molecule protein. The CLIP molecule protein may be CD74, for instance, as discussed above. In the case that CD74 is produced it is desired that the CD74 be produced in the cell such that it can be processed intracellularly to produce a CLIP associated with MHC. Alternatively it may be processed in other cells that are capable of secreting it such that CD74 protein is capable of interacting with MHC on the surface. The expression vector may also produce a CLIP peptide either intracellularly or extracellularly. CLIP activators include for instance exogenous CD74 (such as SEQ ID NO. 2) exogenous CLIP (such as SEQ ID NO. 1), functionally active fragments of CD74 or CLIP (i.e. an amino acid sequence comprising 9 or more consecutive amino acids of SEQ ID NO. 2 or 6 or more consecutive amino acids of SEQ ID NO: 2), palmitoylated protein or PAM, and an anti-CD40 or CD40L molecule in combination with IL-4.

According to an embodiment of the invention, the methods described herein are useful in inhibiting the development of an autoimmune disease such as a gastrointestinal autoimmune disease in a subject by administering a CLIP inducer to the subject. Thus, the methods are useful for such autoimmune diseases as Crohn's disease, ulcerative colitis, inflammatory bowel disease, and celiac disease.

“Autoimmune Disease” refers to those diseases which are commonly associated with the nonanaphylactic hypersensitivity reactions (Type II, Type III and/or Type IV hypersensitivity reactions) that generally result as a consequence of the subject's own humoral and/or cell-mediated immune response to one or more immunogenic substances of endogenous and/or exogenous origin. Such autoimmune diseases are distinguished from diseases associated with the anaphylactic (Type I or IgE-mediated) hypersensitivity reactions. The methods of the invention are particularly useful in the treatment of gastrointestinal autoimmune diseases but are also useful in treating systemic autoimmune diseases that have a gastrointestinal component, such as scleroderma, as long as the therapy is delivered to the gut.

The CLIP inducers can also be administered orally to a subject having a gastrointestinal disorder such as that caused by infections. For instance H. pylori and HIV infection both cause damage to the gastrointestinal tract. Such damage can be avoided by administering to subjects infected with H. pylori or HIV a CLIP inducer. The therapeutic agent is delivered mucosally (orally is preferred for delivery to the gastrointestinal system), such that it is exposed to the cells of the gastrointestinal tract. Systemic therapy for infections such as H. pylori and HIV involve administering an inhibitor of CLIP. Therefore, while the subject is administered an oral dose of a CLIP inducer, the subject may also be treated systemically with a CLIP inhibitor. In some embodiments the CLIP inducer is administered prior to, concurrently with, or following administration of one or more other agents to treat peptic ulcer. Agents to treat peptic ulcer include but are not limited to antibiotics useful for killing H. pylori, such as amoxicillin, clarithromycin (BIAXIN®), metronidazole (FLAGYL®) and tetracycline; medications that block acid production and promote healing, such as omeprazole (PRILOSEC®), lansoprazole (PREVACID®), rabeprazole (ACIPHEX®), esomeprazole (NEXIUM®) and pantoprazole (PROTONIX); medications to reduce acid production, for instance histamine (H-2) blockers, such as ranitidine (ZANTAC®), famotidine (PEPCID®), cimetidine (TAGAMENT®) and nizatidine (AXID®); antacids that neutralize stomach acid; and medications that protect the lining of the stomach and small intestine, for instance, cytoprotective agents such as sucralfate (CARAFATE®) and misoprostol (CYTOTEC®) and bismuth sub salicylate (PEPTO-BISMOL®).

Compositions including combinations of CLIP inhibitors and agents to treat peptic ulcers are also provided according to aspects of the invention. The invention also includes kits which include both a CLIP inhibitor and an agent to treat peptic ulcers as well as instructions for administering the combination of agents.

The invention involves methods for treating a subject. A subject shall mean a human or vertebrate mammal including but not limited to a dog, cat, horse, goat and primate, e.g., monkey. Thus, the invention can also be used to treat diseases or conditions in non human subjects. Preferably the subject is a human.

As used herein, the term treat, treated, or treating when used with respect to a disorder refers to a prophylactic treatment which increases the resistance of a subject to development of the disease or, in other words, decreases the likelihood that the subject will develop the disease as well as a treatment after the subject has developed the disease in order to fight the disease, prevent the disease from becoming worse, or slow the progression of the disease compared to in the absence of the therapy.

When used in combination with the therapies of the invention the dosages of known therapies may be reduced in some instances, to avoid side effects.

The CLIP inhibitor or inducer can be administered in combination with other therapeutic agents and such administration may be simultaneous or sequential. When the other therapeutic agents are administered simultaneously they can be administered in the same or separate formulations, but are administered at the same time. The administration of the other therapeutic agent, including chloroquine and hydroxychloroquine and the CLIP inhibitor or inducer can also be temporally separated, meaning that the therapeutic agents are administered at a different time, either before or after, the administration of the CLIP inhibitor or inducer. The separation in time between the administration of these compounds may be a matter of minutes or it may be longer.

The cancer may also be a mesothelioma which is treated with a combination of an autophagy inhibitor and an anti-VEGF antibody.

According to one set of embodiments, the cells are exposed to an autophagy inhibitor. An “autophagy modulator,” as used herein, is a lysosomotropic agent, meaning that it accumulates preferentially in the lysosomes of cells in the body and blocks pathways involved in break down of cellular components. An autophagy inhibitor, as used herein, is any compound which blocks the collection or metabolism of lipids in the lysosome. The inhibitor is effective for killing cells by inhibiting autophagy in cells that depend on autophagy to survive. While no one knows exactly the mechanism by which autophagy inhibitors function, it may well be through the inhibition of the acidic hydrolases (enzymes in the lysosomes) that are necessary to break down proteins, lipids, etc. for processing and removal by increasing the pH to decrease the necessary acidity for the enzymes to work.

In some embodiments, the autophagy inhibitor is selected from the group consisting of: chloroquine compounds, 3-methyladenine, bafilomycin Al, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels, adenosine, N6-mercaptopurine riboside, wortmannin, and vinblastine.

The autophagy inhibitor is preferably a chloroquine compound. Chloroquine is a synthetically manufactured drug containing a quinoline nucleus (The Merck Index, p. 2220, 1996). The chloroquine compounds useful according to the invention include chloroquine analogs and derivatives. A number of chloroquine analogs and derivatives are well known. For example, suitable compounds include but are not limited to chloroquine, chloroquine phosphate, hydroxychloroquine, chloroquine diphosphate, chloroquine sulphate, hydroxychloroquine sulphate, quinacrine, primaquine, mefloquine, halofantrine, lumefantrine and tafenoquine or enantiomers, derivatives, analogs, metabolites, pharmaceutically acceptable salts, and mixtures thereof.

Chloroquine and hydroxychloroquine are generally racemic mixtures of (−)- and (+)-enantiomers. The (−)-enantiomers are also known as (R)-enantiomers (physical rotation) and 1-enantiomers (optical rotation). The (+)-enantiomers are also known as (S)-enantiomers (physical rotation) and r-enantiomers (optical rotation). Preferably, the (−)-enantiomer of chloroquine is used. The enantiomers of chloroquine and hydroxychloroquine can be prepared by procedures known to the art.

The compounds of the invention, such as, chloroquine may exhibit the phenomena of tautomerism, conformational isomerism, geometric isomerism, and/or optical isomerism. The invention covers any tautomeric, conformational isomeric, optical isomeric and/or geometric isomeric forms of the compounds described herein, as well as mixtures of these various different forms.

Thus in some embodiments the autophagy inhibitor useful in the invention is a 4-aminoquinoline. 4-aminoquinolines include compounds having the following structure:

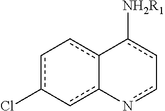

or a pharmaceutically acceptable salt or prodrug thereof, wherein R₁ is defined herein;

each instance of the dotted line independently represents a single bond or a double bond which can be in the cis or trans configuration; and

R₁ is 1 or 2 hydrogens, alkyl, cycloalkyl, aryl, substituted alkyl, substituted cycloalkyl or substituted aryl.

In other embodiments the 4-aminoquinoline has the following structure:

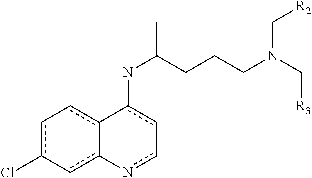

or a pharmaceutically acceptable salt or prodrug thereof, wherein R₂ and R₃ are defined herein;

each instance of the dotted line independently represents a single bond or a double bond which can be in the cis or trans configuration;

R₂ and R₃ is independently a hydroxalkyl, an alkyl, alkyloxy, alkylcarboxy, alkylene or alkenylene having from one to six carbon atoms.

Examples of 4-aminoquinolines useful according to the invention include but are not limited to chloroquine, 2-hydroxychloroquine, amodiaquine, mondesethylchloroquine, quinoline phosphate, and chloroquine phosphate or mixtures thereof.

Choloroquine:

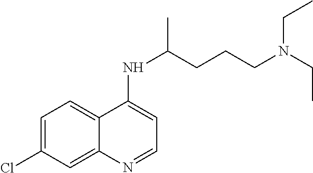 [SMILES: CCN(CC)CCCC(C)Nc1ccnc2cc(Cl)ccc12]

2-hydroxychloroquine

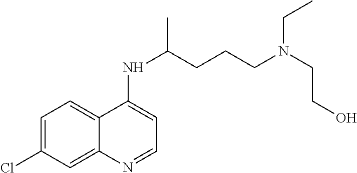 [SMILES: CCN(CCO)CCCC(C)Nc1ccnc2cc(Cl)ccc12]

Other examples of preferred chloroquine compounds that can be used in the invention include chloroquine diphosphate and hydroxychloroquine (PLAQUENIL™)

Anti-VEGF antibodies, as used herein, refer to peptides that bind to VEGF with sufficient affinity and specificity to prevent VEGF from interacting with VEGF receptor and reduce VEGF signaling. The term “VEGF” refers to the vascular endothelial cell growth factor, as described by Leung et al. Science, 246:1306 (1989), and Houck et al. MoI. Endocrin., 5:1806 (1991), together with the naturally occurring allelic and processed forms thereof. The term “VEGF” is also used to refer to known truncated forms of the polypeptide. Preferably, the anti-VEGF antibody of the invention can be used as a therapeutic agent in targeting and interfering with diseases or conditions wherein the VEGF activity is involved. An anti-VEGF antibody will usually not bind to other VEGF homologues such as VEGF-B or VEGF-C, nor other growth factors such as PIGF, PDGF or bFGF. A preferred anti-VEGF antibody is a recombinant humanized anti-VEGF monoclonal antibody generated according to Presta et al. (1997) Cancer Res. 57:4593-4599, including but not limited to the antibody known as bevacizumab (AVASTIN®).

Bevacizumab is a major drug developed for treating cancer, including metastatic cancer, and has the trade name AVASTIN®, by Genentech/Roche. Bevacizumab is a humanized monoclonal antibody, and was the first commercially available angiogenesis inhibitor. It stops tumor growth by preventing the formation of new blood vessels (angiogenesis) by targeting and inhibiting the function of a natural protein called vascular endothelial growth factor that stimulates new blood vessel formation. The drug was first developed as a genetically engineered version of a mouse antibody that contains both human and mouse components, a monoclonal antibody against VEGF-A.

In some embodiments the VEGF antibody is not VEGFR-3 mAb disclosed by Imclone Systems Inc., New York, N.Y.

Numerous VEGF and VEGF receptor antibodies are available commercially for research purposes. Certain portions of the variable domains differ extensively in sequence among antibodies and are used in the binding and specificity of each particular antibody for its particular antigen. However, the variability is not evenly distributed throughout the variable domains of antibodies. It is concentrated in three or four CDRs or “hypervariable regions” in both in the light-chain and the heavy-chain variable domains, as discussed above.

The invention also involves methods of treating disorders with a combination of the CLIP inhibitor and MHC binding molecules as well as an autophagy inhibitor. Thus in some embodiments the invention involves methods of treating breast cancer by administering to the subject, either separately or at the same time, a combination of a CLIP inhibitor, an MHC binding molecule, an autophagy inhibitor and optionally taxane.

The invention also involves a method for treating a subject by administering to a subject having a disease selected from the group consisting of Chronic Lyme, Chronic neuroborreliosis, Multiple sclerosis, Pediatric acute neuropsychiatric disorders associated with infection (i.e. streptococcal, Chronic hepatitis B, Chronic hepatitis C), Burkitt's lymphoma, Epstein bar virus-associated tumors, Systemic lupus Erythematosos, Rheumatoid arthritis, Autoimmune myocarditis, Asthma, Alopecia greata, Anti-phospholipid antibody syndrome, Autoimmune hepatitis, Autoimmune lymphoproliferative syndrome, Goodpasture's syndrome, Myasthenia gravis, Ankylosing spondylitis, Autoimmune hemolytic anemia, Cold agglutin disease, Dermatitis herpiteforms, Dermamyosis, Discoid lupus, Grave's disease, Guillain-Barre syndrome, Hashimoto's disease, IgA nephropathy, Type I diabetes, Meniere disease, MCTD, Polyarteritis nodose, Polymyalgia rheumatic, Reiter's syndrome, Sarcoidoisis, Scleroderma, Sjogreia Syndrome, Wegener's Granulomatosis, and Vitiligo an autophagy inhibitor, a CLIP inhibitor and an MHC binding molecule.

The active agents of the invention are administered to the subject in an effective amount for treating disorders such as autoimmune disease and cancer. An “effective amount”, for instance, is an amount necessary or sufficient to realize a desired biologic effect. An effective amount for treating autoimmune disease may be an amount sufficient to prevent or inhibit a decrease in T_{H} cells compared to the levels in the absence of treatment. According to some aspects of the invention, an effective amount is that amount of a compound of the invention alone or in combination with another medicament, which when combined or co-administered or administered alone, results in a therapeutic response to the disease, either in the prevention or the treatment of the disease. The biological effect may be the amelioration and or absolute elimination of symptoms resulting from the disease. In another embodiment, the biological effect is the complete abrogation of the disease, as evidenced for example, by the absence of a symptom of the disease.

The effective amount of a compound of the invention in the treatment of a disease described herein may vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment.

Multiple doses of the molecules of the invention are also contemplated. In some instances, when the molecules of the invention are administered with another therapeutic, for instance, an anti-cancer agent a sub-therapeutic dosage of either or both of the molecules may be used. A “sub-therapeutic dose” as used herein refers to a dosage which is less than that dosage which would produce a therapeutic result in the subject if administered in the absence of the other agent.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

The agent may comprise different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences (1990), incorporated herein by reference). In a particular embodiment, intraperitoneal injection is contemplated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds of the invention may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

According to the methods of the invention, the compound may be administered in a pharmaceutical composition. In general, a pharmaceutical composition comprises the compound of the invention and a pharmaceutically-acceptable carrier. Pharmaceutically-acceptable carriers for peptides, monoclonal antibodies, and antibody fragments are well-known to those of ordinary skill in the art. As used herein, a pharmaceutically-acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In yet other embodiments, the preferred vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”, claiming priority to U.S. patent application serial no. 213,668, filed Mar. 15, 1994). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Therapeutic formulations of the peptides or antibodies may be prepared for storage by mixing a peptide or antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The peptide may be administered directly to a cell or a subject, such as a human subject alone or with a suitable carrier. Alternatively, a peptide may be delivered to a cell in vitro or in vivo by delivering a nucleic acid that expresses the peptide to a cell. Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, lipo some-mediated transfection, and the like. For certain uses, it is preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid molecule delivery vehicle. Especially preferred are monoclonal antibodies. Where liposomes are employed to deliver the nucleic acid molecules of the invention, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

The peptide of the invention may also be expressed directly in mammalian cells using a mammalian expression vector. Such a vector can be delivered to the cell or subject and the peptide expressed within the cell or subject. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the α-fetoprotein promoter.

As used herein, a “vector” may be any of a number of nucleic acid molecules into which a desired sequence may be inserted by restriction and ligation for expression in a host cell. Vectors are typically composed of DNA although RNA vectors are also available. Vectors include, but are not limited to, plasmids, phagemids and virus genomes. An expression vector is one into which a desired DNA sequence may be inserted by restriction and ligation such that it is operably joined to regulatory sequences and may be expressed as an RNA transcript.

The invention also includes articles, which refers to any one or collection of components. In some embodiments the articles are kits. The articles include pharmaceutical or diagnostic grade compounds of the invention in one or more containers. The article may include instructions or labels promoting or describing the use of the compounds of the invention.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of infections, cancer, and autoimmune disease.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

### Effects of LPS, MKR.4 and Anti-MHC Class II Antibody Treatment on CRL-5822 Cells

In order to determine whether the displacement of CLIP with peptide in the absence or presence of a TLR ligand resulted in induction of Class II associated death in CRL-5822 the following experiment was performed.

Methods

The media was removed from a flask of CRL-5822 using a sterile glass pipet and vacuum and 1.5 mL of accutase was added to the flask and placed in incubator for 20 minutes. The cells were washed with 3 mL of PBS and transferred to a sterile 15 mL conical tube and centrifuged for 5 minutes @ 1000 rpm. The supernatant was removed with sterile glass pipet and vacuum and the pellet resuspended in 5 mL of PBS. A cell count was performed using the TC-10 automated counter and 10 ul of cell suspension+10 ul of trypan blue was loaded into disposable counting slide and put in the TC-10. The following count was made: Live: 3.54*10⁴ cells/mL*5 mL=177,000 cells total & 177,000/9 wells (8 wells in experiment+1 to maintain culture)=19,666 cells/well (flask).

The 5 mL of cell suspension was centrifuged for 5′ at 1000 rpm, the pellet was resuspended in 9 mL of 10% FBS RPMI. 1 mL of the cell suspension was added to a T-25 flask that had 4 mL of media in it and placed in the incubator. After culture 1 mL of the cell suspension was added to each well (8 wells in a labeled 24 well plate). A fresh vial of MKR.4 was resuspended in sterile DMSO. 7 mg of peptide was used to make a 5 mg/mL stock (7 mg/mL=5 mg/mL=1.4 mL of DMSO). The peptide was added into 1.4 mL of DMSO, under the hood to maintain sterility. Once all of the peptide was dissolved, 200 ul aliquots were made.

8 wells were set up for the experiment with no treatment, with LPS, with MKR.4 peptide, with MKR.4 peptide+LPS and with or without addition of purified Anti Class-II (HLA-DR,DP,DQ) treatment in the amounts listed below.

Stock Concentrations:

LPS: 1 ug/mL (Sigma-Aldrich, St. Louis, Mo.)

MKR.4: 2.5 ug/mL (SEQ ID NO 8: ANS GFR IMA VLA SGG QY)

LPS: (x) (5 mg/mL stock)=(1 ug/mL) (1 mL)×=0.2 ul*8 wells=1.6 ul+8.4 ul PBS=10 ul/8 wells=1.25 ul mixture/well

MKR.4: (x) (5 mg/mL stock)=(2.5 ug/mL) (1 mL) x=0.5 ul*4 wells=2 ul MKR.4+8 ul PBS=2.5 ul mixture/well

Anti MHC Class II (eBioscience, San Diego, Calif.) Treatment was at 5 ug/mL Stock was at 1 mg/mL (x) (1 mg/mL)=(5 ug/mL) (1 mL) x=5 ul/well

Treatment Procedure:

LPS was administered to activate the cells and induce CLIP expression. MKR.4 was added to select wells 24 hours after cell activation. The cells were incubated with MKR.4 for 2 hours. Then fresh media with or without Anti MHC Class II was added. 24 hours after Anti MHC Class II was added to the wells viability counts were performed.

Results:

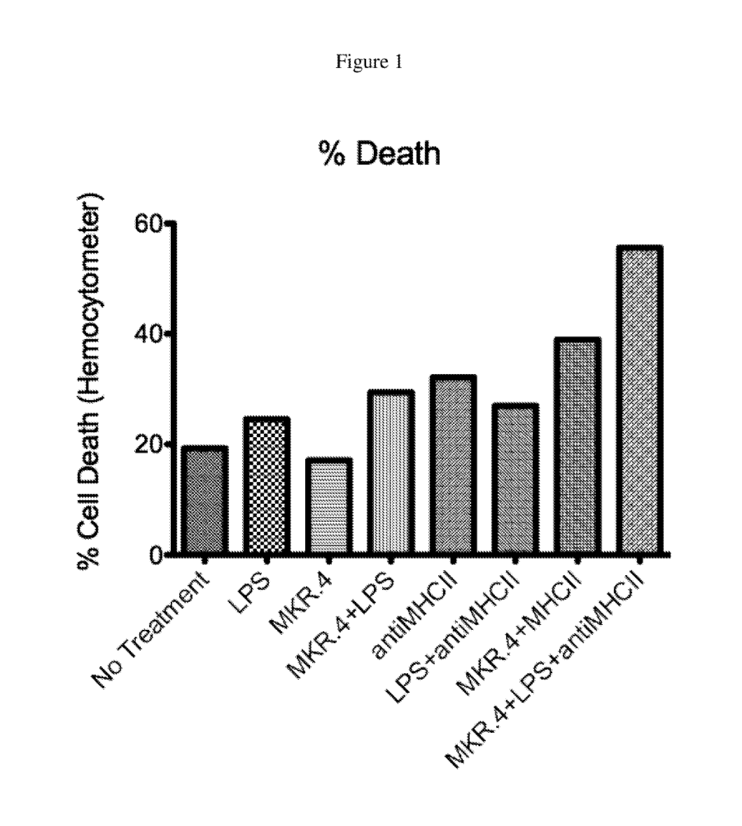

The cell counts are presented below. The results were incorporated into a graph and depicted in FIG. 1.

Counts

| Treatment | Viability results | Live/Dead Calculations |
|---|---|---|
| No Treatment 1:2 in 1 mL | 8/0 11/4 14/2 13/5 | Total Live: 0.23 * 10⁶ |
| | | Total Dead: 0.06 *10⁶ |
| LPS 1:2 in 1 mL | 9/2 12/5 12/1 10/6 | Total Live: 0.22 * 10⁶ |
| | | Total Dead: 0.07 *10⁶ |
| MKR.4 1:2 in 1 mL | 15/3 13/5 22/3 13/2 | Total Live: 0.32 * 10⁶ |
| | | Total Dead: 0.07 *10⁶ |
| MKR.4 + LPS 1:2 in 1 mL | 10/5 17/7 8/2 13/6 | Total Live: 0.24 * 10⁶ |
| | | Total Dead: 0.10 *10⁶ |
| No Treatment + Anti Class | 8/6 10/5 11/4 9/3 | Total Live: 0.19 * 10⁶ |
| II 1:2 in 1 mL | | Total Dead: 0.09 *10⁶ |
| MKR.4 + Anti Class II 1:2 | 14/5 16/10 21/514/4 | Total Live: 0.33 * 10⁶ |
| in 1 mL | | Total Dead: 0.12 *10⁶ |
| LPS + Anti Class II 1:2 in | 11/9 20/6 10/7 | Total Live: 0.29 * 10⁶ |
| 1 mL | 17/15 | Total Dead: 0.19 *10⁶ |
| LPS + MKR.4 + Anti Class | 4/5 3/3 5/7 4/5 | Total Live: 0.08 * 10⁶ |
| II 1:2 in 1 mL | | Total Dead: 0.10 *10⁶ |

### Example 2

### Effects of CpG, MKR.4, and Anti MHC Class II Antibody Treatment on C57BL/6 Splenocytes and CRL-5822 Cells

In order to determine whether the displacement of CLIP with peptide in the absence or presence of a TLR ligand resulted in induction of Class II associated death in C57BL/6 Splenocytes and CRL-5822 the following experiment was performed.

Methods:

CRL-5822 were Prepared as Follows:
Removed the media from a flask of CRL-5822 using a sterile glass pipet and vacuum
Added 1.5 mL of accutase to the flask and placed in incubator for 20 minutes
Washed with 3 mL of PBS
Transferred all to a sterile 15 mL conical tube
Centrifuged for 5′ @ 1000 rpm
Removed supernatant with sterile glass pipet and vacuum
Resuspended in 3 mL of PBS
Performed a hemacytometer count indicating: 1.14*10⁶ cells total of CRL-5822 cells
1.14*10⁶/8 wells+2 wells for culture maintenance=10 wells
   1.14*10⁶/10 wells=11,400 cells per well
Centrifuged the 3 mL of cell suspension for 5′ at 1000 rpm
Resuspended pellet in 10 mL of 10% FBS RPMI
1 mL of this was added to 2 wells in a 12-well plate added 1 mL of fresh 10% FBS RPMI to each well to bring the total volume up to 2 mL.

C57BL/6 were Prepared as Follows:
A spleen from a C57BL/6 mouse was placed in a cell strainer in a petri dish with 6 mL of PBS
The spleen was mashed to produce a cell suspension
The PBS splenocyte cell suspension was transferred into a labeled 15 mL conical tube and centrifuged for 5 minutes at 1000 rpm
The pellet was resuspended in 1 mL of PBS
Geys was prepared to lyse the red blood cells
   7 mL of DI H20;
   0.5 mL of Geys B
   0.5 mL of Geys C
   weighed out 0.07 mg of NH4Cl
   Mixed the 0.07 mg of NH4Cl with 2 mL of Geys A
   Transferred the dissolved solution to a 15 mL conical tube with the H20, Geys B, and Geys C
3 mL of the Geys solution was added to the 1 mL of PBS cell suspension, mixed well and placed on ice for 1 minute (frequently inverted solution in tube on ice during the 1 minute)
The mixture was centrifuged for 5 minutes at 1000 rpm, washed with 5 mL of PBS, centrifuged for 5 minutes at 1000 rpm and resuspended in 10 mL of PBS
A hemacytometer count was performed: 99.0*10⁶ cells total (9.9*10⁶ cells/mL) plate 4*10⁶ cells per well*8 wells=32 million; 32*10⁶/9.9*10⁶=3.23 mL of this suspension needed to obtain 32*10⁶ cells
Centrifuged 3.23 mL of the suspension for 5 minutes at 1000 rpm and resuspended the pellet in 8 mL of 5% FBS RPMI
1 mL of suspension was added to each well (12-well plate)
Added another 2 mL of 5% FBS RPMI to each well to bring the final volume up to 3 mL

8 wells were set up for the experiment for each cell type (CRL-5822 and C57BL/6) with no treatment, with CpG, MKR.4 peptide, with MKR.4 peptide+CpG and with or without addition of purified Anti Class-II (HLA-DR,DP,DQ) treatment in the amounts listed below.

Stock Concentrations:

CPG: 1 mg/mL (SEQ ID NO 9: 5′-tcgtcgttttgtcgttttgtcgtt-3′, purchased from
In Vitrogen)
MKR.4: 5 mg/mL (SEQ ID NO 8: ANS GFR IMA VLA SGG QY)

CRL-5822
CpG:(x) (1 mg/mL stock)=(1 ug/mL) (2 mL)x=2 ul
MKR.4: (x) (5 mg/mL stock)=(2.5 ug/mL) (2 mL) x=1 ul
Anti MHC Class II: Treat at 5 ug/mL; Stock is at 1 mg/mL; (x) (1 mg/mL)=(5 ug/mL) (2 mL); x=10 μl/well

C57BL/6

CpG: (x) (1 mg/mL stock)=(5 ug/mL) (3 mL) x=15 ul

MKR.4: (x) (5 mg/mL stock)=(5 ug/mL) (3 mL) x=3 ul

Anti MHC Class II: Treat at 5 ug/mL; Stock is at 1 mg/mL; (x) (1 mg/mL)=(5 ug/mL) (3 mL); x=15 ul/well

Treatment Procedure

CpG was administered to activate the cells and induce CLIP expression. MKR.4 was added to select wells 24 hours after cell activation. The cells were incubated with MKR.4 for 2 hours. Then fresh media with or without Anti MHC Class II was added. 24 hours after Anti MHC Class II was added to the wells viability counts were performed.

Results:

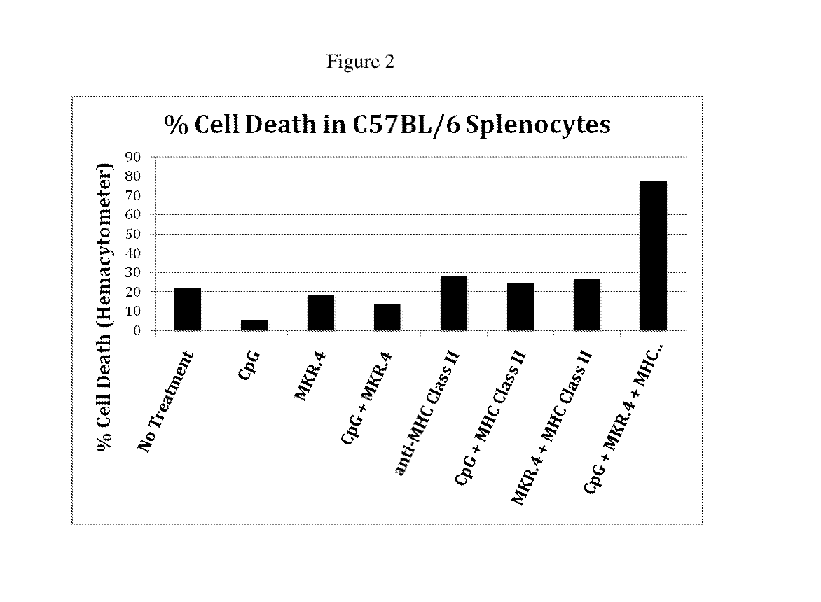

The cell counts are presented below. The results for C57BL/6 were incorporated into a graph and depicted in FIG. 2.

CRL-5822 Counts

| Treatment | Viability results | Live/Dead Calculations |
|---|---|---|
| No Treatment 1:2 in 1 mL | 9/4 19/4 7/2 10/3 | Total Live: 0.23 * 10⁶ |
| | | Total Dead: 0.07 *10⁶ |
| CpG 1:2 in 1 mL | 12/6 21/3 24/3 13/2 | Total Live: 0.35 * 10⁶ |
| | | Total Dead: 0.07 *10⁶ |
| MKR.4 1:2 in 1 mL | 13/3 10/4 12/2 12/7 | Total Live: 0.24 * 10⁶ |
| | | Total Dead: 0.08 *10⁶ |
| MKR.4 + CpG 1:2 in 1 mL | 10/5 10/4 13/4 17/4 | Total Live: 0.25 * 10⁶ |
| | | Total Dead: 0.09 *10⁶ |
| No Treatment + Anti Class | 5/14 8/18 6/13 7/22 | Total Live: 0.13 * 10⁶ |
| II 1:2 in 1 mL | | Total Dead: 0.34 *10⁶ |
| MKR.4 + Anti Class II 1:2 | 5/18 4/12 13/12 5/13 | Total Live: 0.14 * 10⁶ |
| in 1 mL | | Total Dead: 0.28 *10⁶ |
| CpG + Anti Class II 1:2 in | 20/1 21/5 16/2 15/6 | Total Live: 0.36 * 10⁶ |
| 1 mL | | Total Dead: 0.07 *10⁶ |
| CpG + MKR.4 + Anti Class | 15/10 16/8 9/10 5/7 | Total Live: 0.23 * 10⁶ |
| II 1:2 in 1 mL | | Total Dead: 0.18 *10⁶ |

C57BL/6 Counts

| Treatment | Viability results | Live/Dead Calculations |
|---|---|---|
| No Treatment 1:5 in 1 | 13/3 13/3 13/411/4 | Total Live: 0.63 * 10⁶ |
| mL | | Total Dead: 0.18 *10⁶ |
| CpG 1:5 in 1 mL | 20/0 28/3 31/2 21/1 | Total Live: 1.25 * 10⁶ |
| | | Total Dead: 0.08 *10⁶ |
| MKR.4 1:2 in 1 mL | 21/5 26/616/5 24/4 | Total Live: 0.44 * 10⁶ |
| | | Total Dead: 0.10 *10⁶ |
| MKR.4 + CpG 1:5 in | 26/2 34/4 18/3 30/8 | Total Live: 1.35 * 10⁶ |
| 1 mL | | Total Dead: 0.21 *10⁶ |
| No Treatment + Anti | 28/9 27/12 19/8 31/13 | Total Live: 0.53 * 10⁶ |
| Class II 1:2 in 1 mL | | Total Dead: 0.21 *10⁶ |
| MKR.4 + Anti Class II | 10/3 11/2 12/6 10/5 | Total Live: 0.22 * 10⁶ |
| 1:2 in 1 mL | | Total Dead: 0.08 *10⁶ |
| CpG + Anti Class II | 12/4 10/5 18/8 24/4 | Total Live: 0.80 * 10⁶ |
| 1:5 in 1 mL | | Total Dead: 0.26 *10⁶ |
| CpG + MKR.4 + Anti | 6/13 4/24 8/153/18 | Total Live: 0.26 * 10⁶ |
| Class II 1:5 in 1 mL | | Total Dead: 0.88 *10⁶ |

### Example 3

### Effects of CpG, MKR.4 and Anti-MHC Class II Antibody Treatment on H69 or WITT Cells

Methods: Cells:

Based on cell counts 500,000 H69 cells per well were set up in a 6 well plate-4.0*10⁶/2.95*10⁶=1.36 mL. The 1.36 mL was added to a new 15 mL conical tube and centrifuged for 5 minutes at 1000 rpm. The pellet was resuspended in 8 mL of H69 media and 1 mL of cell suspension was added to each well. 2 mL of H69 media was added to each well to bring the total volume/well up to 3 mL and the plates were placed in an incubator.

0.59*10⁶ cells/mL*5 mL=2.93*10⁶ of WITT cells total were used. The cells were centrifuged at 1000 rpm for 5 minutes and the pellet was resuspended in 9 mL of WITT media. 1 mL of this cell suspension was added to each of 12 wells and a culture flask. Another 2 mL of WITT media was added to each well in the 12 well plate to bring the total volume up to 3 mL. Another 9 mL was added to the T-25 culture flask to bring that total volume up to 10 mL. The plate and flask were placed in the incubator to allow adherence overnight.

Treatment Procedure:

CpG was administered to activate the cells and induce CLIP expression. 15 uL of 1 mg/mL CpG was added into each CpG treated well. MKR.4 was added to select wells 24 hours after cell activation. The cells were incubated with 3 uL of 5 mg/mL stock MKR.4 in DMSO for 2 hours. Then fresh media with or without Anti MHC Class II was added (5 ul/well of stock at 1 mg/mL). 24 hours after Anti MHC Class II was added to the wells viability counts were performed.

Staining:

16 treatment groups with 3 stains per treatment group=48 treatments+2 accounting for error=50 staining solutions. For 50 stains at 50 uL, 2,500 uL of PBS and 50 uL of anti-human CLIP were used. The following anti-human CLIP antibody: BD Pharmingen Cat #555981 Lot#86069 was used.

Results:

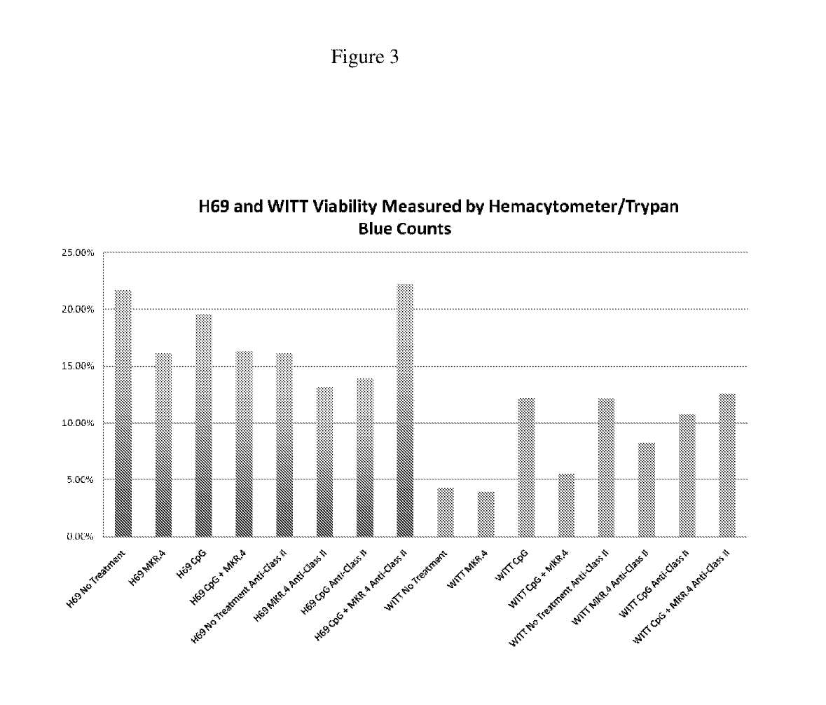

The cell counts are presented below for each of H69 and WITT. The results were incorporated into a graph and depicted in FIG. 3.

Viability Counts Using Hemacytometer:

H69Cells

| Treatment | Viability results | Live/Dead Calculations |
|---|---|---|
| No Treatment 1:10 in | 13/3 21/5 16/4 15/6 | Live: 1.63*10⁶ |
| 1 mL | | Dead: 0.45*10⁶ |
| MKR.4 1:5 in 1 mL | 34/9 29/8 23/2 31/3 | Live: 1.46*10⁶ |
| | | Dead: 0.28*10⁶ |
| CpG 1:10 in 1 mL | 13/4 14/5 19/2 20/5 | Live: 1.65*10⁶ |
| | | Dead: 0.40*10⁶ |
| CpG + MKR.4 1:5 in | 19/5 26/2 11/1 26/8 | Live: 1.03*10⁶ |
| 1 mL | | Dead: 0.20*10⁶ |
| No Treatment + anti- | 26/4 31/6 22/5 29/6 | Live: 1.35*10⁶ |
| class II 1:5 in 1 mL | | Dead: 0.26*10⁶ |
| MKR.4 + anti-class II | 26/4 23/4 31/4 30/5 | Live: 1.38*10⁶ |
| 1:5 in 1 mL | | Dead: 0.21*10⁶ |
| CpG + anti-class II 1:5 | 21/4 24/6 21/2 23/2 | Live: 1.11*10⁶ |
| 1 mL | | Dead: 0.18*10⁶ |
| CpG + MKR.4 + anti- | 13/4 22/9 11/2 21/4 | Live: 0.84*10⁶ |
| class II 1:5 1 mL | | Dead: 0.24*10⁶ |

WITT Cells

| Treatment | Viability results | Live/Dead Calculations |
|---|---|---|
| No Treatment 1:2 in 1 mL | 54/1 53/146/3 48/4 | Live: 1.01*10⁶ Dead: 0.045*10⁶ |
| MKR.4 1:5 in 1 mL | 17/1 26/0 17/1 14/1 | Live: 0.93*10⁶ Dead: 0.038*10⁶ |
| CpG 1:5 in 1 mL | 13/2 23/1 18/3 21/4 | Live: 0.94*10⁶ Dead: 0.13*10⁶ |
| CpG + MKR.4 1:5 1 mL | 25/3 22/0 24/0 15/2 | Live: 1.08*10⁶ Dead: 0.063*10⁶ |
| No Treatment + anti-class II 1:5 in 1 mL | 24/2 20/3 25/4 24/4 | Live: 1.16*10⁶ Dead: 0.16*10⁶ |
| MKR.4 + anti-class II 1:5 in 1 mL | 19/1 20/2 15/0 24/4 | Live: 0.98*10⁶ Dead: 0.088*10⁶ |
| CpG + anti-class II 1:5 in 1 mL | 24/2 14/213/1 15/3 | Live: 0.83*10⁶ Dead: 0.1*10⁶ |
| CpG + MKR.4 + anti-class II 1:5 in 1 mL | 19/4 30/3 1/2 23/4 | Live: 1.11*10⁶ Dead: 0.16*10⁶ |

FIG. 3 depicts % cell death in either untreated H69 or WITT cells or H69 or WITT cells treated with CpG, MKR.4, MKR.4+CpG, anti-MHC class II antibody, CpG+anti-MHC class II antibody, MKR.4+anti-MHC class II antibody or MKR.4+CpG+anti-MHC class II antibody (cell viability depicted as hemacytometer/trypan blue counts).

### Example 4

### Effects of CpG, MKR.2, MKR.4, and Anti-MHC Class II Antibody Treatment on L1210DDP Cells

Methods: Cells:

3.25*10⁶ cells/mL*5 mL=16.25*10⁶ of L1210DDP cells total were used. The cells were centrifuged at 1000 rpm for 5 minutes and the pellet was resuspended in media. 1 mL of this cell suspension was added to each of 14 wells. The total well volume up to 2 mL.

Treatment Procedure:

The treatment was performed as described in Example 3, with the addition of MKR.2 (SKM RMA TPL LMQ ALY, SEQ ID NO: 76)

Staining:

5 staining groups (No Stain, Iso CLIP, CLIP, CLIP, CLIP) with 0.5*10⁶ cells per sample were set up for baseline staining. 0.5*10⁶ cells*5 samples=2.5*10⁶/3.25*10⁶=769 ul. 769 ul of cell suspension was removed and stained with antibody. The following anti-mouse CLIP antibody: Santa Cruz Cat# sc-53946 Lot# G1807 was used.

Results:

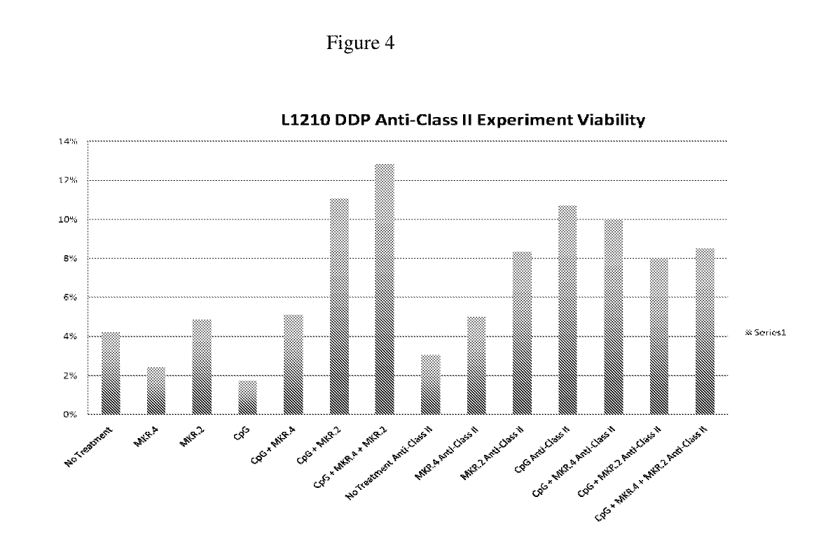

The cell counts are presented below. The results were incorporated into a graph and depicted in FIG. 4.

Staining Post Treatment: The following anti-mouse CLIP antibody: Santa Cruz Cat# sc-53946 Lot# G1807 was used.

| Treatment | Viability results | Live/Dead Calculations |
|---|---|---|
| No Treatment 1:20 in 1 mL | 30/1 20/3 38/048/2 | Live: 6.80*10\hat{ }6 Dead: 0.3*10\hat{ }6 |
| MKR.4 1:20 in 1 mL | 35/2 39/1 41/1 45/0 | Live: 8.00*10\hat{ }6 Dead: 0.20*10\hat{ }6 |
| MKR.2 1:20 in 1 mL | 26/0 33/0 26/4 52/3 | Live: 6.85*10\hat{ }6 Dead: 0.35*10\hat{ }6 |
| CpG 1:20 in 1 mL | 12/0 16/0 12/1 17/0 | Live: 2.85*10\hat{ }6 Dead: 0.05*10\hat{ }6 |
| CpG + MKR.4 1:20 in 1 mL | 79/4 97/6 71/3 87/5 | Live: 16.7*10\hat{ }6 Dead: 0.90*10\hat{ }6 |
| CpG + MKR.2 1:20 in 1 mL | 39/3 45/4 44/8 33/5 | Live: 8.05*10\hat{ }6 Dead: 1.00*10\hat{ }6 |
| CpG + MKR.4 + MKR.2 1:20 in 1 mL | 19/3 36/4 32/4 42/8 | Live: 6.45*10\hat{ }6 Dead: 0.95*10\hat{ }6 |
| No Treatment + anti-class II 1:20 in 1 mL | 18/1 36/2 15/0 27/0 | Live: 4.80*10\hat{ }6 Dead: 0.15*10\hat{ }6 |
| MKR.4 + anti-class II 1:20 in 1 mL | 36/3 37/1 29/331/0 | Live: 6.65*10\hat{ }6 Dead: 0.35*10\hat{ }6 |
| MKR.2 + anti-class II 1:20 in 1 mL | 14/0 25/3 15/2 23/2 | Live: 3.85*10\hat{ }6 Dead: 0.35*10\hat{ }6 |
| CpG + anti-class II 1:20 in 1 mL | 18/1 22/0 13/5 22/3 | Live: 3.75*10\hat{ }6 Dead: 0.45*10\hat{ }6 |
| CpG + MKR.4 + anti-class II 1:20 in 1 mL | 34/3 24/4 29/2 30/4 | Live: 5.85*10\hat{ }6 Dead: 0.65*10\hat{ }6 |
| CpG + MKR.2 + anti-class II 1:20 in 1 mL | 18/1 19/1 19/2 25/3 | Live: 4.05*10\hat{ }6 Dead: 0.35*10\hat{ }6 |
| CpG + MKR.4 + MKR.2 + anti-class II | 29/4 27/2 13/0 17/2 | Live: 4.30*10\hat{ }6 Dead: 0.40*10\hat{ }6 |
| 1:20 in 1 mL | | |

FIG. 4 is a bar graph depicting % cell death in either untreated L1210DDP cells or L1210DDP cells treated with CpG, MKR.4, MKR.2, MKR.4+CpG, MKR.2+CpG, MKR.4+MKR.2+CpG, anti-MHC class II antibody, CpG+anti-MHC class II antibody, MKR.4+anti-MHC class II antibody, MKR.2+anti-MHC class II antibody, MKR.4+CpG+anti-MHC class II antibody, MKR.2+CpG+anti-MHC class II antibody, or MKR.4+MKR.2+CpG+anti-MHC class II antibody.

### Example 5

### Effects of LPS, MKR.4 and Anti-MHC Class II Antibody Treatment on CRL-5822 Cells

In order to examine the effects of estrogen treatment on cancer cell CLIP expression, a drug sensitive cell line (MCF7) and a drug resistant cell line (MCF7-ADR) of human breast cancer were treated with b-estradiol (estrogen) and tested for changes in CLIP expression.

MCF7 (ATCC (cat# HTB-22)) and MCF7-ADR were grown in 10% FBS RPMI (Gibco RPMI 1640 media cat #21870076) supplemented with Fetal Bovine Serum, L-Glutamine, 2-Mercaptophenol, Hepes Buffer, Sodium Pyruvate, and Gentamicin).

1.07*10⁶MCF7 cells were plated per well in 2 wells. 1.11 MCF7 ADR cells were plated per well in 2 wells. The cells were treated with B-estradiol from Sigma (Cat# E2758-250 mg, Lot#110M0138V). In order to prepare a 10 nM final concentration 27.238 mg of B-estradiol was weighed under the hood with a sterile weigh boat and spatula and dissolved in 1 mL of DMSO to make a 0.1M stock, which was then diluted twice:
1:1000 dilution (1 ul of the 0.1M stock to 999 ul PBS)
1:100 dilution (10 ul of the 1:000 dilution to 990 ul PBS)

20 ul of the 1:100 diluted solution was added to a final volume of 2 mL per treatment well. The MCF7 and MCF7 ADR were harvested about 19 hours after treatment. The cells were counted and viability is shown below.

| Treatment | Viability results | Live/Dead Calculations |
|---|---|---|
| MCF7 No Treatment 1:2 in 1 mL | 23/6 31/6 20/4 24/5 | Live: 0.49*10⁶ Dead: 0.11*10⁶ |
| MCF7 B-Estradiol 1:2 in 1 mL | 26/6 31/2 15/9 28/5 | Live: 0.50*10⁶ Dead: 0.11*10⁶ |
| MCF7 ADR No Treatment 1:2 in 1 mL | 28/1 22/2 16/3 20/5 | Live: 0.43*10⁶ Dead: 0.06*10⁶ |
| MCF7 ADR B-Estradiol 1:2 in 1 mL | 35/2 18/4 24/4 16/4 | Live: 0.47*10⁶ Dead: 0.07*10⁶ |

Staining:

After counting the cells were centrifuged at 1000 rpm for 5 minutes. The pellet was resuspended in 250 ul of PBS and 50 ul of this solution was aliquoted into a labeled 96 well plate. 50 ul of antibody mixture (calculations below using a repeating pipettor) was added and the plate was placed in refrigerator for 15 minutes. 100 ul of PBS was used to wash each well. The plate was centrifuged at 1000 rpm for 5 minutes and the supernatant was flicked off. Each well was resuspended into 100 ul of PBS and 200 ul of PBS was added to flow tubes. 100 ul of cell suspension from each well was added into the sample tubes.

Antibody calculation: 4 treatment groups and 3 stains per treatment group
4*3=12+2 error=14
Human antibodies use 3 ul/stain so 14*3=42 ul antibody 14 stains*50 uL=700 uL of PBS−42 uL of anti-human CLIP BD (Pharmingen, Cat #555981, Lot#86069)
658 uL PBS+42 uL anti-human CLIP

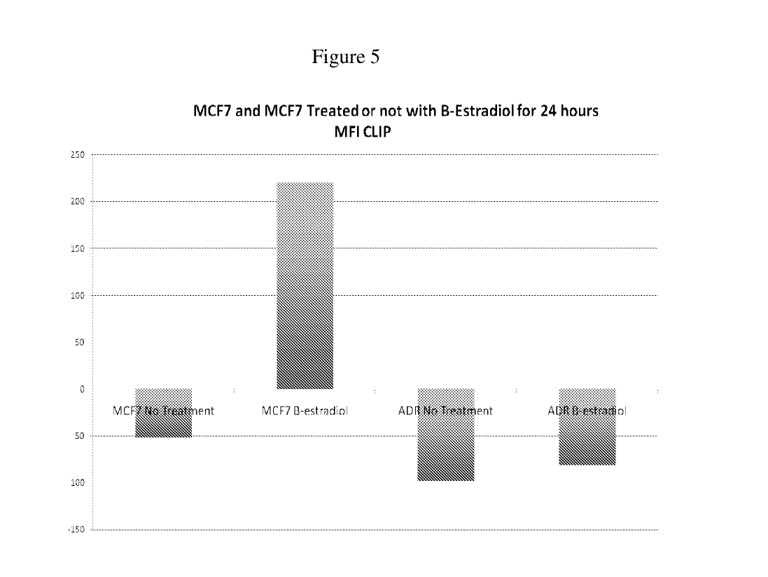

The results are shown in FIG. 5. The drug sensitive cell line (MCF7) responded to the b-estradiol and resulted in increased CLIP expression. Untreated MCF7 cells and estradiol treated and untreated drug resistant MCF7 cells (ADR) did not induce CLIP expression. The drug resistant cell line (MCF7 ADR) does not have estrogen receptors, and therefore, was not affected by the addition of B-estradiol for this experiment.

### Example 6

### Effects of CpG, MKR.4, and Anti MHC Class II Antibody Treatment on Murine Splenocytes and CLIP−/−Mice

In order to determine whether the displacement of CLIP with peptide in the absence or presence of a TLR ligand resulted in induction of Class II associated death in C57BL/6 Splenocytes and CLIP−/− mice the following experiment was performed.

Methods:

B cells were isolated from the spleens 12 week old male C57B/6 or CLIP−/− (Invariant Chain deficient) mice (Jackson Labs) using the Miltenyi Biotech B cell isolation kit according to the manufacturer's directions. Cells were incubated with or without CpG-ODN (1 mg/mL SEQ ID NO 9: 5′-tcgtcgttttgtcgttttgtcgtt-3′, purchased from Invivogen) for 48 hours followed by treatment with 5 μg/mL MKR.4 (5 mg/mL, SEQ ID NO 8: ANS GFR IMA VLA SGG QY). Cells were incubated with MKR.4 for 1 hour and then washed followed by treatment with 2.5 μg/mL anti-MHCII (IA/IE, BD Bioscience). 24 hours after anti-MHCII treatment cells were analyzed for cell death using Acridine Organge/propidium iodide staining measured on the Cellometer (Nexcelom), propidium iodide and foreword Vs. side scatter staining measured on a BD FACS Canto II flow cytometer (BD Bioscience). Data was analyzed using FlowJo software (TreeStar).

Results:

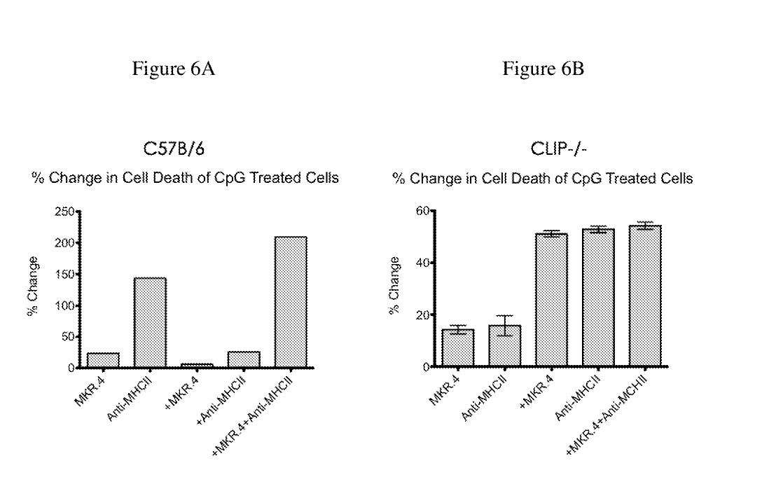

The results are shown in FIG. 6. FIG. 6A depicts the percent change in cell death in B cells isolated from splenoctyes of C57B/6 mice. The results for the B cells isolated from the CLIP−/− mice are shown in FIG. 6B. The percent change refers to the percent increase in cell death. The lanes having the + before the treatment indicate pre-treatment with CpG ODN. As demonstrated in the data, the presence of CLIP is protective of the CpG stimulated cell. For instance, in normal cells that are not CpG stimulated, treatment with MHC class II antibody results in an increase in cell death. When the cells are stimulated with CpG the increased cell death resulting from treatment with MHC class II antibody is blocked. However, the addition of the CLIP inhibitor (last lane) restores the increase in cell death. In the CLIP−/−cells, CLIP is not able to provide a protective function for the cells and the introduction of the CLIP inhibitor has no effect on the cell death caused by the MHC class II antibody.

### Example 7

### Effects of CpG, MKR.4, RT2 and Anti MHC Class II Antibody Treatment on Murine Splenocytes and Daudi Cells

In order to determine whether the displacement of CLIP with peptide in the absence or presence of a TLR ligand resulted in induction of Class II associated death in C57BL/6 Splenocytes and Daudi cells the following experiments were performed.

Methods:

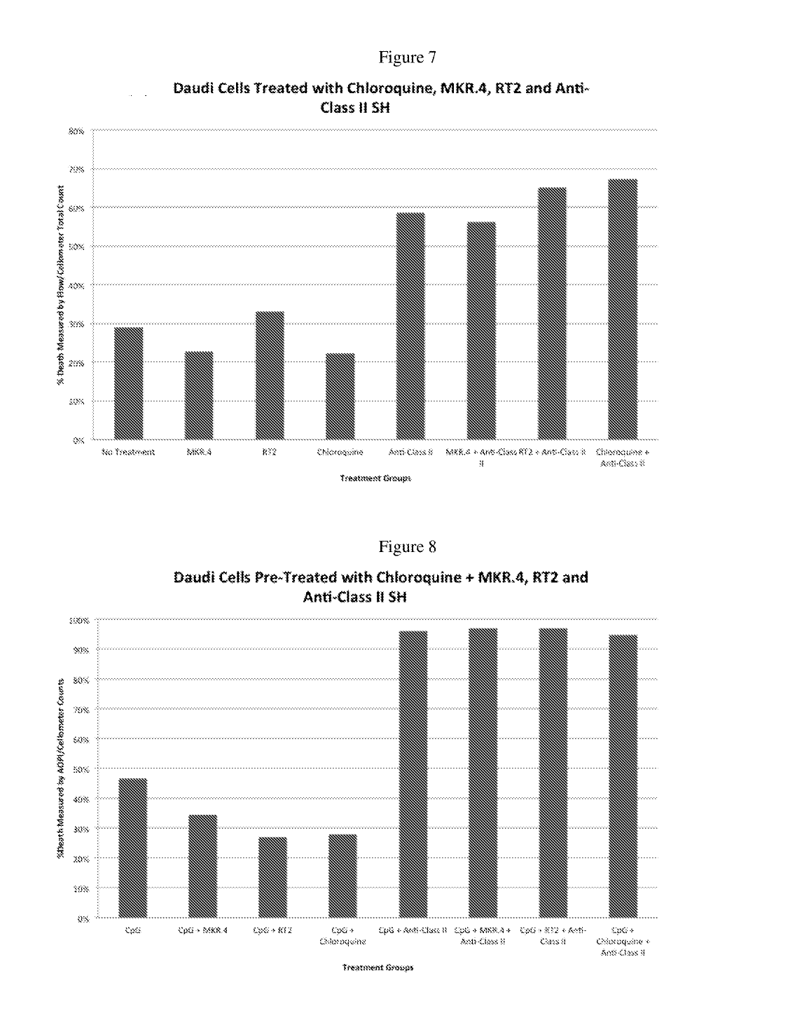

Daudi cells (ATCC) were treated with MKR.4 (SEQ ID NO 8, Viral Genetics) at 5 ug/mL, RT2 (a low binder for MHC class II: ENM LRS MPV KGK RKD; Viral Genetics) at 5 ug/mL, Hydroxychloroquine (Sigma-Aldrich) at 0.1 mM and incubated for 24 hours with these treatments in a 5% CO2 incubator. Following 24 hours, Purified azide-free Mouse Anti-Human HLA-DR, DQ, DQ (BD Pharmingen) was added at 2.5 ug/mL. The cells were incubated another 24 hours in a 5% CO2 incubator following addition of the Rat Anti-Mouse I-A/1-E. The cells were then analyzed for cell death utilizing AO/PI staining solution (Nexcelom Biosciences) and a Cellometer Counter (Nexcelom Biosciences) (FIG. 7). In another set of experiments the Daudi cells were pre-treated with CpG ODN (SEQ ID NO 9, Invivogen) at 2 ug/mL and then treated as described above (FIG. 8).

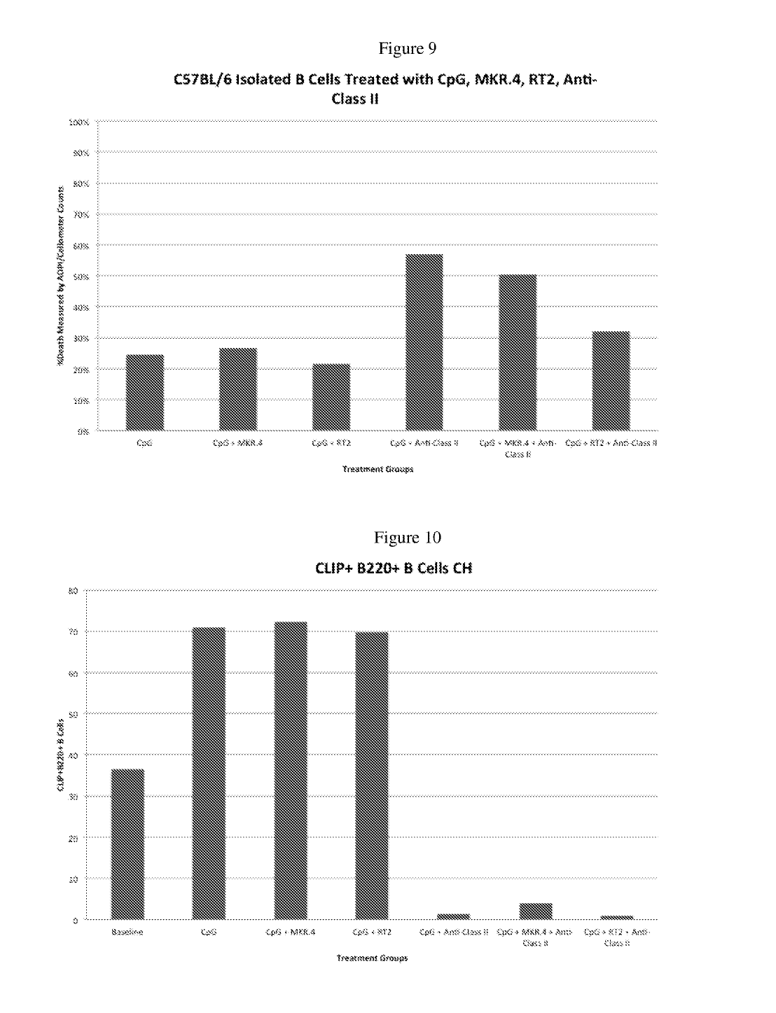

Splenocyte cells were collected from a C57BL/6 male at 10 weeks of age (Jackson Laboratories). A B-cell isolation was performed to obtain a population of purified B-cells utilizing a B-cell isolation kit (Miltenyi Biotech). The isolated B-cells were then treated with CpG ODN (SEQ ID NO 9, Invivogen) at 2.5 ug/mL. These were incubated for 48 hours in a 5% CO2 incubator. The B-cells were then treated with MKR.4 (SEQ ID NO 8, Viral Genetics) at 5 ug/mL, RT2 (Viral Genetics) at 5 ug/mL. Following 24 hours, purified azide-free Mouse Anti-Human HLA-DR, DQ, DQ (BD Pharmingen) was added at 2.5 ug/mL. The cells were incubated another 24 hours in a 5% CO2 incubator following addition of the Rat Anti-Mouse I-A/I-E. The cells were then analyzed for cell death utilizing AO/PI staining solution (Nexcelom Biosciences) and a Cellometer Counter (Nexcelom Biosciences). (FIG. 9). In separate experiments, cells were treated as described above for FIG. 9 and then stained for CD45R (BD Pharmingen) and CLIP (Santa Cruz Biotech). The samples were run by a BD FACSCanto II flow cytometer (BD Biosciences). The data was analyzed by FlowJo (Treestar) (FIG. 10).

Results:

The results are shown in FIGS. 7-10. As shown in FIGS. 7 and 8 the addition of anti-MHC class II antibody to the treated cells resulted in increased cell death in the presence (FIG. 8) or absence of CpG ODN (FIG. 7). The addition of anti-MHC class II antibody to treated splenocytes also resulted in an increase in cell death as shown in FIG. 9. Cells treated according to the protocols of FIG. 9 were stained for CD45R (BD Pharmingen) and CLIP (Santa Cruz Biotech) as shown in FIG. 10.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method of treating a subject having cancer, comprising administering to the subject an isolated MHC class II specific CLIP inhibitor and an MHC binding agent in an effective amount to treat the subject.

2. The method of claim 1, wherein the cancer is a gastrointestinal cancer, a glioblastoma, a colon carcinoma, or a pancreatic cancer.

3-4. (canceled)

5. The method of claim 1, wherein the MHC binding agent is an anti-MHC class II antibody.

6-11. (canceled)

12. A method of treating a subject having a hyperproliferative disease, comprising administering to the subject an isolated MHC class II specific CLIP inhibitor and an MHC binding agent in an effective amount to treat the subject.

13-14. (canceled)

15. A method of treating a subject having a hormone positive breast or prostate cancer, comprising administering to the subject an isolated MHC class II specific CLIP inhibitor and an MHC binding agent in an effective amount to treat the subject.

16-19. (canceled)

20. A method of treating a subject, comprising identifying a subject having an MHC positive cancer and administering to the subject a CLIP inhibitor and an MHC binding agent in an effective amount to treat the subject.

21-30. (canceled)

31. A method of inducing MHC class I or MHC class II-mediated cell death, comprising contacting an MHC expressing cell, wherein the MHC expressing cell is not a B cell, with a CLIP inhibitor in an effective amount to remove endogenous CLIP peptide from the cell surface and contacting the cell with an MHC binding agent to induce MHC class I or MHC class II-mediated cell death.

32-43. (canceled)

44. A method of protecting a cell from MHC class I or MHC class II-mediated cell death, comprising contacting an MHC expressing cell, wherein the MHC expressing cell is not a B cell, with a CLIP inducing agent in an effective amount to place CLIP peptide on the cell surface and protect the cell from MHC class I or MHC class II-mediated cell death.

45-58. (canceled)

59. The method of claim 44, wherein the subject has an autoimmune disease and the CLIP inducing agent is administered to the subject orally.

60. The method of claim 59, wherein the autoimmune disease is Crohn's disease.

61. The method of claim 59, wherein the autoimmune disease is ulcerative colitis.

62. A method of treating a subject having mesothelioma, comprising administering to the subject an autophagy inhibitor and an anti-VEGF antibody in an effective amount to treat the subject.

63. The method of claim 62, wherein the anti-VEGF antibody is bevacizumab.

64. The method of claim 62, further comprising administering to the subject a CLIP inhibitor.

65-78. (canceled)
